SEQUENCE LISTING (10 sequences)

SEQ ID NO: 1 - protein, 12 aa, Human immunodeficiency virus 1
YGRKKKRRQRRR

SEQ ID NO: 2 - protein, 18 aa, synthetic construct
LLIILRRRIRKQAHAHSK

SEQ ID NO: 3 - protein, 18 aa, synthetic construct
RRLRRLLRRLRRLLRRLR

SEQ ID NO: 4 - protein, 16 aa, synthetic construct
RWFKIQMQIRRWKNKK

SEQ ID NO: 5 - protein, 16 aa, synthetic construct
KWFKIQMQIRRWKNKR

SEQ ID NO: 6 - protein, 23 aa, Xenopus laevis
GIGKFLHSAKKFGKAFVGEIMNS

SEQ ID NO: 7 - protein, 16 aa, synthetic construct
  SITE 16: note = amidated
RQIKIWFQNRRMKWKK

SEQ ID NO: 8 - protein, 6 aa, synthetic construct
RRRRRR

SEQ ID NO: 9 - protein, 8 aa, synthetic construct
RRRRRRRR

SEQ ID NO: 10 - protein, 9 aa, synthetic construct
RRRRRRRRR

(19) United States
(12) Patent Application Publication
(10) Pub. No.: US 2024/0415784 A1
(43) Pub. Date: Dec. 19, 2024

(54) MUCOSAL PATCHES AND METHODS OF USE THEREOF

(71) Applicant: Afyx Development A/S, Hørsholm (DK)

(72) Inventor: Martin Eduardo SANTOCILDES ROMERO, Hørsholm (DK)

(21) Appl. No.: 18/712,365

(22) PCT Filed: Nov. 23, 2022
(86) PCT No.: PCT/EP2022/082999

§ 371 Date: May 22, 2024

Related U.S. Application Data
(60) Provisional application No. 63/282,548, filed on Nov. 23, 2021.

(51) Int. Cl. A61K 9/70; A61K 31/58; A61K 47/10; A61K 47/14; A61K 47/32

(52) U.S. Cl. CPC A61K 9/7069; A61K 31/58; A61K 47/10; A61K 47/14; A61K 47/32

(57) ABSTRACT

The present disclosure relates to flexible electrospun patches comprising an electrospun active agent-containing layer and a hydrophobic layer. When applied to a target site. the flexible patches adhere for a time period sufficient to release therapeutically effective amounts of active agent in order to treat a variety of conditions, including vulvar conditions such as vulvar lichen sclerosus.

### CROSS-REFERENCE TO RELATED APPLICATIONS

This application claims the benefit of and priority to U.S. Provisional Application No. 63/282,548, filed Nov. 23, 2021, which is incorporated herein by reference in its entirety.

### BACKGROUND

Mucosal conditions are often difficult to treat with conventional therapeutic compositions and dressings. For example, lichen sclerosus (LS) is a chronic inflammatory skin disease that most often affects the genital (e.g., vulva) and perianal areas. Typical symptoms include skin lesions, as well as pruritus and pain. Therapeutic intervention in the form of topical creams and ointments is generally required for improvement. Yet, even with these therapies, the condition can persist for years and recurrence is common.

Accordingly, there is a need in the art for new therapeutic compositions that provide more effective treatment of mucosal conditions, including inflammatory skin conditions such as vulvar lichen sclerosus.

### SUMMARY

To address the aforementioned issues, the present disclosure provides flexible, optionally active agent-containing electrospun patches, with an impermeable layer, that are designed for adhesion to a targeted site, and when containing an active agent, provide localized, unidirectional delivery of the active agent to the targeted site, e.g., mucosa or skin. These flexible patches, which were found to be easy to apply and comfortable to wear, adhere to the targeted area for prolonged periods of time. Combined with high encapsulation efficiency and rapid release rates of the active agent, in various embodiments the disclosed patches are able to controllably release an active agent to the affected area at a clinically effective rate.

Accordingly, in some embodiments, the patches disclosed herein are useful in treating a variety of diseases and conditions, including chronic inflammatory skin conditions, such as vulvar lichen sclerosus.

In one aspect, the present disclosure provides a patch with improved flexibility for treating a vulvar disease or condition, comprising: (a) an electrospun fiber layer, wherein the electrospun fiber layer comprises electrospun fibers comprising: one or more polymers; and a therapeutically effective amount of an active agent, and (b) an impermeable layer, wherein after application to a patient in need thereof, the patch provides a therapeutically effective amount of the active agent for at least about 2 hours and wherein the patch stiffness is from about 250 mN/mm to about 450 mN/mm, as determined by a three-point bend test.

In one aspect, the present disclosure provides a patch with improved flexibility for treating an oral disease or condition, comprising: (a) an electrospun fiber layer, wherein the electrospun fiber layer comprises electrospun fibers comprising: one or more polymers; and a therapeutically effective amount of an active agent, and (b) an impermeable layer, wherein after application to a patient in need thereof, the patch provides a therapeutically effective amount of the active agent for at least about 2 hours and wherein the patch stiffness is from about 250 mN/mm to about 450 mN/mm, as determined by a three-point bend test.

In some embodiments, at least one of the electrospun fibers and the impermeable layer further comprises a plasticizer. In some embodiments, the electrospun fibers further comprise a plasticizer. In some embodiments, the impermeable layer comprises a plasticizer.

In some embodiments, the plasticizer is selected from the group consisting of citrate esters, fatty acid esters, sebacate esters, phthalate esters, and glycol derivatives. In some embodiments, the plasticizer is selected from the group consisting of triethyl citrate, acetyl triethyl citrate, tributyl citrate, butyl stearate, glycerol monostearate, stearyl alcohol, castor oil, mineral oil, diacetylated monoglycerides, dibutyl sebacate, diethyl phthalate, dibutyl phthalate, dioctyl phosphate, sorbitol, glycerol, triacetin, tributyrin, cellulose nitrate, polyethylene glycol, polyethylene glycol monomethyl ether, propylene glycol, or mixtures thereof. In some embodiments, the plasticizer is dibutyl sebacate.

In some embodiments, the plasticizer is present in an amount ranging from about 5.0% to about 30% by weight of the patch.

In some embodiments, the electrospun fibers comprise about 1% to about 20% by weight of the plasticizer. In some embodiments, the impermeable layer comprises about 1% to about 20% by weight of the plasticizer

In some embodiments, the one or more polymers are selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the one or more polymers are selected from the group consisting of polyvinylpyrrolidone (PVP), acrylates and acrylic copolymers and mixtures thereof. In some embodiments, the one or more polymers are polyvinylpyrrolidone (PVP), an ammonio methacrylate copolymer type B, or mixtures thereof. In some embodiments, the weight ratio of polyvinylpyrrolidone to ammonio methacrylate copolymer is about 0.8 to about 2. In some embodiments, the electrospun fiber layer further comprises a bioadhesive substance. In some embodiments, the bioadhesive substance is selected from the group consisting of dextran, polyethylene oxide (PEO), alginate, tragacanth, carrageenan, pectin, gelatin, guar, xanthan, gellan, methylcellulose, hydroxypropylmethylcellulose (HPMC), carboxymethylcellulose and alkali salts thereof, polymers of acrylic acids (PAA derivatives), chitosan, lectins, thiolated polymers, polyox WSR, PAA-co-PEG (PEG is polyethylene glycol), and mixtures thereof. In some embodiments, the bioadhesive substance is PEO. In some embodiments, the weight ratio of PVP: ammonio methacrylate copolymer B: PEO is from about 2:1: 1 to about 2:2.5:4

In some embodiments, the area density of the electrospun fiber layer is less than about 125 grams per square meter (gsm). In some embodiments, the area density of the electrospun fiber layer is less than about 100 grams per square meter (gsm). In some embodiments, the area density of the electrospun fiber layer is less than about 75 gsm. In some embodiments, the area density of the electrospun fiber layer is from about 15 gsm to about 75 gsm. In some embodiments, the area density of the electrospun fiber layer is greater than the impermeable layer.

In some embodiments, the impermeable layer comprises a hydrophobic polymer. In some embodiments, the hydrophobic polymer is selected from the group consisting of the group consisting of polyethylene-co-vinyl acetate, ethylcellulose, poly (caprolactone) (PCL), carbothane or polysoftane. In some embodiments, the hydrophobic polymer is PCL.

In some embodiments, the vulvar disease or condition is selected from the group consisting of vulva lichen sclerosus, vulva lichen planus, vulva psoriasis, vulva dermatosis and vulva lichen simplex chronicus. In some embodiments, the vulvar disease or condition is vulva lichen sclerosus.

In some embodiments, the active agent is an anti-inflammatory agent or an immunosuppressant. In some embodiments, the anti-inflammatory agent or immunosuppressant is a corticosteroid. In some embodiments, the corticosteroid is selected from the group consisting of diflorasone diacetate, betamethasone dipropionate, mometasone furoate and clobetasol propionate. In some embodiments, the corticosteroid is mometasone furoate. In some embodiments, the immunosuppressant is a calcineurin inhibitor. In some embodiments, the calcineurin inhibitor is cyclosporine, tacrolimus, or pimecrolimus.

In some embodiments, the electrospun fiber layer comprises about 0.01% to about 50.0% (wt./wt. %) of the active agent. In some embodiments, the electrospun fiber layer comprises about 1.0% to about 10.0% (wt./wt. %) of the active agent.

In some embodiments, at least about 50% of the active agent is released from the electrospun fiber layer within about 30 min after application. In some embodiments, complete active agent release from the electrospun fiber layer is achieved about 2-3 h after application.

In some aspects, the present disclosure provides a method of treating an oral disease or condition in a patient in need thereof, the method comprising applying a patch disclosed herein to oral mucosa of a patient in need thereof.

In some aspects, the present disclosure provides a method of treating a vulvar disease or condition in a patient in need thereof, the method comprising applying a patch disclosed herein to a vulvar lesion on a patient in need thereof.

In some aspects, the present disclosure provides a method of treating vulvar lichen sclerosus in a patient in need thereof, the method comprising applying a patch disclosed herein to a VLS lesion on a patient in need thereof.

In some embodiments, the patch is applied once a day, twice a day, three times a day or four times a day. In some embodiments, the patch is applied once a day.

### BRIEF DESCRIPTION OF THE DRAWINGS

FIG. 1 shows the amount of mometasone (as a percent of mass of electrospun fiber material) recovered from samples of electrospun fibers in methanol. Sample 1:0. 1% by weight mometasone; Sample 2:0.01% by weight mometasone; Sample 3:0. 1% by weight mometasone/7.35% by weight dibutyl sebacate; Sample 4:0.01% by weight mometasone/7.35% by weight dibutyl sebacate.

FIG. 2 shows the amount of mometasone release from electrospun fibers (Samples 1-4) over three hours in phosphate-buffered saline as the amount of mometasone released as percent of mass of electrospun fibers (FIG. 2A) and the percent of total amount of released mometasone (FIG. 2B).

FIG. 3A provides a graph comparing the force applied to biadhesive electrospun monolayer compositions with (Modified) and without (Rivelin) dibutyl sebacate plasticizer.

FIG. 3B provides a graph comparing the force applied to biadhesive electrospun dual-layer compositions with (Modified) and without (Rivelin) dibutyl sebacate plasticizer.

### DETAILED DESCRIPTION

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . “, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

“Optional” or “optionally” means that the subsequently described element, event or circumstances may or may not occur, and that the description includes instances where said event, element, or circumstance occurs and instances in which it does not.

As used herein, “patient” include vertebrates, more specifically a mammal (e.g., a human, horse, pig, rabbit, dog, sheep, goat, non-human primate, cow, cat, guinea pig or rodent), a fish, a bird or a reptile or an amphibian. In some embodiments, the patient is a human patient. Unless otherwise specified, the term patient does not denote a particular age or sex. In some embodiments, the patient may be a geriatric patient, a pediatric patient, a teenage patient, a young adult patient, or a middle-aged patient. In some embodiments, the patient is less than about 18 years of age. In some embodiments, the patient is at least about 18 years of age. In some embodiments, the patient is about 5-10, about 10-15, about 15-20, about 20-25, about 25-30, about 30-35, about 35-40, about 40-45, about 45-50, about 50-55, about 55-60, about 60-65, about 65-70, about 70-75, about 75-80, about 85-90, about 90-95, or about 95-100 years of age. In some embodiments, the patient is a male patient. In some embodiments, the patient is a female patient. In some embodiments, the patient is diagnosed with or is considered at risk of having or developing a vulvar disease or condition, e.g., vulvar lichen sclerosus.

As used herein, the term “impermeable layer” or “impermeable backing layer” refers to a coating or barrier layer in a layered patch configuration that prevents or substantially limits the passage of water through the layer. The impermeable layer can also prevent or substantially limit the passage of the active ingredient(s) through the layer.

As used herein, the term “oligomeric plasticizer” refers to a polymeric plasticizer having a lower molecular weight than plasticizers considered to be “polymeric”. Typically, oligomeric plasticizers have up to 100 repeating units.

As used herein, the term “polymeric plasticizer” refers to a polymeric plasticizer having greater than 100 repeating units. In some embodiments, the polymeric plasticizer of the present disclosure has a molecular weight (MW) up to about 100,000 Da. In some embodiments, the polymeric plasticizer has a molecular weight up to about 35,000 Da. In some embodiments, the polymeric plasticizer has a molecular weight from about 2,000 Da to about 100,000 Da. In some embodiments, the polymeric plasticizer has a molecular weight from about 2,000 Da to about 35,000 Da.

As used herein, the term “treating” with regard to a patient, refers to improving at least one symptom of the patient's disorder. Treating can be curing, improving, or at least partially ameliorating a disorder. The term “therapeutic effect” as used herein refers to a desired or beneficial effect provided by the method and/or the composition. For example, the method for treating a disclosed disease or condition provides a therapeutic effect when the method reduces at least one symptom of the disease or condition in a patient.

As used herein, the term “preventing” with regard to a patient refers to reducing or eliminating the onset of the symptoms or complications of a disease, condition or disorder. In some embodiments, the symptoms or complications are reduced or eliminated in a patient that is predisposed to the disease, condition, or disorder. The patches of the present disclosure are capable of preventing or treating such diseases, condition, or disorders.

All documents cited herein are incorporated by reference in their entirety for all purposes.

Inflammatory anogenital skin conditions such as vulvar, penile, and perianal lichen sclerosus can result in painful lesions and significant discomfort that if left untreated may cause permanent damage. Topical ointments and creams are commonly prescribed as treatment, but these formulations often can be inconvenient to apply to the affected area, and may need reapplication one or more times throughout the day. This is particularly problematic as conditions like lichen sclerosus tend to recur, meaning that prolonged and repetitive periods of treatment may be required.

Flexible and optionally active agent-containing patches may provide an alternative option for treating inflammatory anogenital skin conditions. However, existing oral patches (see International Publication Nos. WO2015/189212, WO2017/085264, WO2018/133910, and WO2018/133909; U.S. Pat. No. 10,052,291; and U.S. Publication Nos. 2019/0254985, 2019/0254986, and 2019/0351662 describing highly mucoadhesive oral patches that attach to mucosal surfaces and deliver an active agent, e.g., the anesthetic lidocaine or the steroid clobetasol propionate, which are hereby incorporated by reference in their entireties for all purposes) may not have suitable properties to deliver therapeutically effective amounts of active agent to the anogenital area.

To address the issues, the present disclosure describes optionally active agent-containing electrospun patches with increased flexibility (or lower stiffness) that in various embodiments are able to provide localized, unidirectional delivery of therapeutically effective amounts of active agent to the genital and perianal area. As described herein, it was discovered that patch flexibility (or stiffness) could be tuned by the addition of plasticizer (described herein), by adjusting the composition of the one or more polymers in the electrospun fiber, and/or by varying patch area density.

The disclosed flexible patches adhere to the anogenital region of a subject and were found to be comfortable for prolonged periods of time after application. These properties enable long residence times of the disclosed patches at the treatment site that can be effective in treating inflammatory skin conditions (e.g., vulvar lichen planus) and other diseases that affect the external genitals and perianal area, particularly when combined with high encapsulation efficiency and clinically useful release rates of the active agent.

In some embodiments, the present disclosure provides a patch for treating a disease or condition, comprising: (i) an electrospun fiber layer, wherein the electrospun fiber layer comprises electrospun fibers comprising: (a) an active agent; (b) one or more hydrophilic polymers; (c) a bioadhesive substance; and (d) an optional plasticizer; and (ii) a hydrophobic layer, comprising: (a) a hydrophobic polymer; and (b) an optional plasticizer, wherein the patch provides a therapeutically effective amount of active agent for at least about 2 hours after application to a lesion.

The flexibility of the disclosed patches (e.g., as measured by stiffness) can be determined by any method known in the art. In some embodiments, the flexibility of the patches disclosed herein is determined by a three-point bend test, which can be carried out, for example, using a texture analyzer. An example of a suitable three-point bend test is described in Byun et al. The Effect of Calcium on the Cohesive Strength and Flexural Properties of Low-Methoxyl Pectin Biopolymers, Molecules, 2020, 25 (1), 75, the subject matter of which is incorporated herein in its entirety for all purposes.

In some embodiments, the patch of the present disclosure has a stiffness of less than about 650 mN/mm, less than about 625 mN/mm, less than about 600 mN/mm, less than about 575 mN/mm, less than about 550 mN/mm, less than about 525 mN/mm, less than about 500 mN/mm, less than about 475 mN/mm, less than about 450 mN/mm, less than about 425 mN/mm, less than about 400 mN/mm, less than about 375 mN/mm, less than about 350 mN/mm, less than about 325 mN/mm, less than about 300 mN/mm, less than about 275 mN/mm, less than about 250 mN/mm, less than about 225 mN/mm, or less than about 200 mN/mm, as measured by a three-point bend test. In some embodiments, the patch of the present disclosure has a stiffness of less than about 500 mN/mm, as measured by a three-point bend test. In some embodiments, the patch of the present disclosure has a stiffness of less than about 450 mN/mm, as measured by a three-point bend test. In some embodiments, the patch of the present disclosure has a stiffness of less than about 400 mN/mm, as measured by a three-point bend test. In some embodiments, the patch of the present disclosure has a stiffness of from about 200 mN/mm to about 650 mN/mm, e.g., about 200 mN/mm, about 250 mN/mm, about 300 mN/mm, about 350 mN/mm, about 400 mN/mm, about 450 mN/mm, about 500 mN/mm, about 550 mN/mm, about 600 mN/mm, or about 650 mN/mm, as determined by a three-point bend test. In some embodiments, the patch of the present disclosure has a stiffness of from about 250 mN/mm to about 450 mN/mm, as determined by a three-point bend test. In some embodiments, the patch of the present disclosure has a stiffness of from about 300 mN/mm to about 400 mN/mm, as determined by a three-point bend test. In some embodiments, the patch of the present disclosure has a stiffness of from about 300 mN/mm to about 350 mN/mm, as determined by a three-point bend test.

In some embodiments, the three-point bend test is carried out on a patch having a size and shape that is effective for treating a condition disclosed herein. In some embodiments, the patches subjected to the three-point bend test are circular, semicircular, oval, semioval (i.e., half-moon shaped), square, or rectangular. In some embodiments, the patches are semicircular or semioval. In some embodiments, the patches are semicircular. In some embodiments, the patches are semioval. In some embodiments, a rectangular patch from about 2 cm to about 4 cm in width and from about 5 cm to about 10 cm in length is subjected to the three-point bend test. In some embodiments, a semioval patch from about 2 cm to about 4 cm in width and from about 5 cm to about 10 cm in length is subjected to the three-point bend test.

In some embodiments, the present disclosure provides a patch for treating a disease or condition, comprising: (a) an electrospun fiber layer, wherein the electrospun fiber layer comprises electrospun fibers comprising: one or more polymers; and a therapeutically effective amount of an active agent, and (b) an impermeable layer, wherein after application to a patient in need thereof, the patch provides a therapeutically effective amount of the active agent for at least about 2 hours, and wherein the patch stiffness is from about 250 mN/mm to about 450 mN/mm, as determined by a three-point bend test.

In some embodiments, the flexibility of the patches disclosed herein is determined by measuring patch resilience. As understood in the art, resilience is a measure of the patch's ability to absorb energy when deformed elastically, and then release that energy upon unloading. In some embodiments, the percent resilience of the patch is about 10% to about 50%, e.g., about 10%, about 15%, about 20%, about 25%, about 30%, about 35%, about 40%, about 45%, or about 50%, as determined from a three-point bend test.

In some embodiments, the flexibility of the patches disclosed herein is determined by measuring its stress relaxation. As understood in the art, stress relaxation is the observed decrease in stress in response to a constant strain applied to a material. In some embodiments, stress relaxation refers to a decreased tendency for a material to return to its original shape when unloaded. In some embodiments, the stress relaxation of the patch is about 100 g to about 500 g, e.g., about 100 g, about 125 g, about 150 g, about 175 g, about 200 g, about 225 g, about 250 g, about 275 g, about 200 g, about 325 g, about 350 g, about 375 g, about 400 g, about 425 g, about 450 g, about 475 g, or about 500 g, as determined from a three-point bend test.

In some embodiments, the flexibility of the patches disclosed herein is determined by measuring its burst strength. As understood in the art, burst strength refers to the perpendicular force required to break a material. In some embodiments, the burst strength of the patch is about 2 kg to about 20 kg, e.g., about 2 kg, about 4 kg, about 6 kg, about 8 kg, about 10 kg, about 12 kg, about 14 kg, about 16 kg, about 18 kg, or about 20 kg, as calculated from a three-point bend test.

In some embodiments, the hydrophobic layer comprises one or more plasticizers disclosed herein. In some embodiments, the electrospun fibers comprise one or more plasticizer disclosed herein.

In some embodiments, the one or more polymers comprise polyvinylpyrrolidone, an ammonio methacrylate copolymer type B, and PEO; the impermeable layer comprises PCL, and the plasticizer is dibutyl sebacate.

The electrospun fiber layer and impermeable layer are joined to provide a patch using methods that are known to those skilled in the art, for example, the methods disclosed in International Publication No. WO/2018/133909.

The patch of the present disclosure can be used to treat any disease or condition disclosed herein. In some embodiments, the disease or condition is an inflammatory skin disease of the genital and/or perianal areas (i.e., the anogenital area). In some embodiments, the disease or condition is a vulvar disease or condition. In some embodiments, the disease or condition is vulvar lichen sclerosus, vulva lichen planus, vulva psoriasis, vulva dermatosis and vulva lichen simplex chronicus. In some embodiments, the disease or condition is vulvar lichen sclerosus. In some embodiments, the disease or condition is male genitalia lichen sclerosus. In some embodiments, the disease or condition is penile lichen sclerosus (PLS). In some embodiments, the disease or condition is an oral disease or condition. In some embodiments, the disease or condition is an oral mucosal disease or condition. In some embodiments, the oral disease or condition is selected from the group consisting of recurrent aphthous stomatitis, oral lichen planus, pemphigoid, pemphigus, oral mucositis, and Bechet's disease.

In some embodiments, when the disease or condition is vulvar lichen sclerosus, the lesion is a VLS lesion. In some embodiments, when the disease or condition is penile lichen sclerosus, the lesion is a PLS lesion.

In order to provide effective treatment, the patches of the present disclosure adhere to the targeted site (e.g., vulva, glans penis, or perineum) for a time period sufficient to provide (i.e., release) an effective amount of a therapeutic active agent. Accordingly, in some embodiments, the patch adheres to the targeted site for a period greater than 2 h, greater than 4 h, greater than 8 h, greater than 12 h, greater than 16 h, greater than 24 h, greater than 30 h, greater than 36 h, greater than 42 h, or greater than 48 h. In some embodiments, the patch adheres to the targeted site for a period of at least 2 h. In some embodiments, the patch adheres to the targeted site for a period of at least 4 h. In some embodiments, the patch adheres to the targeted site for a period of at least 6 h. In some embodiments, the patch adheres to the targeted site for a period of at least 8 h. In some embodiments, the patch adheres to the targeted site for a period of at least 12 h. In some embodiments, the patch adheres to the targeted site for a period of at least 24 h.

Without being bound by any particular theory, the impermeable layer, e.g., a hydrophobic impermeable layer, protects the patch from saliva or other sources of moisture in order to prevent it from detaching from the application site (skin or mucosa) prematurely. In addition, the impermeable layer facilitates the application of the patch to the targeted site by providing a non-adhesive surface that will not stick the fingers.

In some embodiments, a patch according to the present disclosure provides a therapeutically effective amount of an active agent for at least about 2 h, at least about 4 h, at least about 6 h, at least about 8 h, at least about 10 h, at least about 12 h, at least about 14 h, at least about 16 h, at least about 18 h, at least about 20 h, at least about 22 h, or at least about 24 h following application to a patient in need thereof. In some embodiments, the patch provides a therapeutically effective amount of an active agent for at least about 4 h following application to a patient in need thereof. In some embodiments, the patch provides a therapeutically effective amount of an active agent for at least about 8 h following application to a patient in need thereof. In some embodiments, the patch provides a therapeutically effective amount of an active agent for at least about 12 h following application to a patient in need thereof.

In some embodiments, a patch according to the present disclosure is applied to a patient in need thereof (for example, the disease site of a patient including, but not limited to, the vulva, glans penis, and perineum) once a day, twice a day (b.i.d.), three times per day (t.i.d.), or four times a day (q.i.d.). In some embodiments, the patch is applied to a patient in need thereof (for example, the disease site of a patient) once a day. In some embodiments, the patch is applied to a patient in need thereof (for example, the disease site of a patient) twice a day. In some embodiments, the patch is applied to a patient in need thereof (for example, the disease site of a patient) three times per day. In some embodiments, the patch is applied to a patient in need thereof (for example, the disease site of a patient) four times per day. In other embodiments, the patch is applied to a patient in need thereof on an as-needed basis.

In some embodiments, a patch according to the present disclosure is applied to a patient in need thereof (for example, the disease site of a patient including, but not limited to, the vulva, glans penis, and perineum) once a day for a period of about 2 to 8 weeks. In some embodiments, the patch is applied to a patient in need thereof (for example, the disease site of a patient) twice a day for a period of about 2 to 8 weeks. In some embodiments, the patch is applied to a patient in need thereof (for example, the disease site of a patient) three times per day for a period of about 2 to 8 weeks. In some embodiments, the patch is applied to a patient in need thereof (for example, the disease site of a patient) four times per day for a period of about 2 to 8 weeks. In some embodiments, the patch is applied to a patient in need thereof (for example, the disease site of a patient) once a day for a period of about 2 to 4 weeks. In some embodiments, the patch is applied to a patient in need thereof (for example, the disease site of a patient) twice a day for a period of about 2 to 4 weeks. In some embodiments, the patch is applied to a patient in need thereof (for example, the disease site of a patient) three times per day for a period of about 2 to 4 weeks. In some embodiments, the patch is applied to a patient in need thereof (for example, the disease site of a patient) four times per day for a period of about 2 to 4 weeks. In some embodiments, the patch is applied to a patient in need thereof (for example, the disease site of a patient) once a day for at least a month. In some embodiments, the patch is applied to a patient in need thereof (for example, the disease site of a patient) twice a day for at least a month. In some embodiments, the patch is applied to a patient in need thereof (for example, the disease site of a patient) three times per day for a period of at least a month. In some embodiments, the patch is applied to a patient in need thereof (for example, the disease site of a patient) four times per day for a period of at least a month. In some embodiments, the patch is applied to a patient in need thereof (for example, the disease site of a patient) once a day for at least a week. In some embodiments, the patch is applied to a patient in need thereof (for example, the disease site of a patient) twice a day for at least a week. In some embodiments, the patch is applied to a patient in need thereof (for example, the disease site of a patient) three times per day for a period of at least a week. In some embodiments, the patch is applied to a patient in need thereof (for example, the disease site of a patient) four times per day for a period of at least a week.

The flexibility of the patches disclosed herein can be adjusted to improve comfort and adherence to the disease site, e.g., the vulva, perineum, or glans penis. In some embodiments, the flexibility of the patch is adjusted by modifying the thickness of the patch, which comprises an electrospun layer and an impermeable layer. In some embodiments, for patches of the same or substantially similar composition, decreasing the thickness of the patch results in an increase in patch flexibility. In some embodiments, the average thickness of the patch prior to application is less than about 1 mm, less than about 0.8 mm, less than about 0.6 mm, or less than about 0.3 mm. In some embodiments, the average thickness of the patch prior to application is less than about 1 mm. In some embodiments, the average thickness of the patch prior to application is less than about 0.6 mm. In some embodiments, the average thickness of the patch prior to application is less than about 0.3 mm.

The patches of the present disclosure can be any shape and size suitable for effective treatment of the affected skin or tissue. In some embodiments, the patches of the present disclosure are circular, semicircular, oval, semioval (i.e., half-moon shaped), square, or rectangular. In some embodiments, the patches are semicircular or semioval. In some embodiments, the patches are semicircular. In some embodiments, the patches are semioval. In some embodiments, a semioval patch is from about 2 cm to about 4 cm in width and from about 5 cm to about 10 cm in length.

### Electrospun Fiber Layer

As used in the present disclosure, the term “electrospun fibers” includes fibers that are obtained by a method that involves electrostatics. The methods, referred in the art as electrohydrodynamic (EHD) methods, include electrospinning, coaxial electrospinning, coaxial electrospraying, emulsion electrospinning, and the like. Any such method can be used to prepare the fibers of the present disclosure. Accordingly, the term “electrospun fibers” is not limited to fibers obtained by electrospinning, but instead refers to fibers obtained by electrohydrodynamic methods known in the art.

In some embodiments, the electrospun fibers or the fiber layer of the patches of the present disclosure comprises about 0.01% to about 50.0% (wt./wt. %), e.g., about 0.01%, about 0.1%, about 0.5%, about 1%, about 2.5%, about 5%, about 7.5%, about 10%, 12.5%, about 15%, about 17.5%, about 20%, 22.5%, about 25%, about 27.5%, about 30%, about 32.5%, about 35%, about 37.5%, about 40%, 42.5%, about 45%, about 47.5%, or about 50%, of the active agent, including all ranges and values therebetween. In some embodiments, the electrospun fiber or fiber layer comprises about 1.0% to about 10.0% (wt./wt. %) of the active agent. In some embodiments, the electrospun fiber or fiber layer comprises about 1.0% to about 5.0% (wt./wt. %) of the therapeutic polypeptide.

In general, the electrospun fibers of the present disclosure are provided in a layer, which when applied to the skin, mucosa or a humid internal surface of the body adhere to the surface. In some embodiments, an active agent (e.g., an anti-inflammatory and/or immunosuppressant active agent) can be homogeneously distributed in the electrospun fibers, whereby the concentration of active agent substance per surface area of the layer is constant and a dose of the active agent substance can easily be determined by using a measured area of the layer.

In some embodiments, the electrospun fiber layer of the present disclosure has an area density less than about 150 grams per square meter (gsm), less than about 140 gsm, less than about 125 gsm, less than about 115 gsm, less than about 105 gsm, less than about 95 gsm, less than about 90 gsm, less than about 85 gsm, less than about 80 gsm, less than about 75 gsm, less than about 70 gsm, less than about 65 gsm, less than about 60 gsm, less than about 55 gsm, less than about 50 gsm, less than about 45 gsm, less than about 40 gsm, less than about 35 gsm, less than about 30 gsm, less than about 25 gsm, less than about 20 gsm, less than about 15 gsm, less than about 10 gsm, or less than about 5 gsm, including all ranges and values therebetween. In some embodiments, the electrospun fiber layer has an area density less than about 150 gsm. In some embodiments, the electrospun fiber layer has an area density less than about 125 gsm. In some embodiments, the electrospun fiber layer has an area density less than about 100 gsm. In some embodiments, the electrospun fiber layer has an area density less than about 75 gsm. In some embodiments, the electrospun fiber layer has an area density less than about 50 gsm. In some embodiments, the electrospun fiber layer has an area density less than about 25 gsm. In some embodiments, the electrospun fiber layer has an area density less than about 15 gsm. In some embodiments, the electrospun fiber layer has an area density of from about 10 gsm to about 100 gsm. In some embodiments, the electrospun fiber layer has an area density of from about 15 gsm to about 75 gsm. In some embodiments, the electrospun fiber layer has an area density of from about 15 gsm to about 50 gsm. In some embodiments, the electrospun fiber layer has an area density of from about 15 gsm to about 30 gsm. In some embodiments, the electrospun fiber layer has an area density of from about 15 gsm to about 20 gsm. In some embodiments, the electrospun fiber layer has an area density of about 17 gsm. In some embodiments, reducing the area density of the electrospun fiber layer results in an increase in the flexibility of the patch. In some embodiments, a patch with increased flexibility is more comfortable after application to the treatment area of a subject in need thereof. In some embodiments, a patch with increased flexibility adheres to the anogenital area for a longer time period compared to a less flexible patch.

In some embodiments, the electrospun fibers of the present disclosure comprise one or more polymers, a therapeutically effective amount of an active agent, and optionally, a plasticizer, each of which is described in more detail below.

In some embodiments, the electrospun fibers of the present disclosure comprise one or more polymers. In some embodiments, the one or more polymers is a hydrophilic polymer, a hydrophobic polymer, or mixture thereof.

In some embodiments, the electrospun fibers of the present disclosure comprise one or more polymers selected from the group consisting of dextran, polyethylene oxides, alginate, tragacanth, carrageenan, pectin, gelatin, guar, xanthan, gellan, fibronectin, collagen, hyaluronic acid, chitosan, cellulosic polymers such as methylcellulose, hydroxypropylmethylcellulose (HPMC), hydroxyethylcellulose, cellulose acetates, carboxymethylcellulose and alkali salts thereof, polymers of acrylic acids (PAA derivatives), chitosan, lectins, thiolated polymers, polyox WSR, PAA-co-PEG (PEG is polyethylene glycol), polylactic acid, polyglycolic acid, poly (butylene succinate), and mixtures thereof. In some embodiments, the electrospun fibers comprise one or more polymers selected from the group consisting of the group consisting of polyethylene-co-vinyl acetate, ethylcellulose, poly (caprolactone) (PCL), carbothane or polysoftane.

In some embodiments, the electrospun fibers of the present disclosure comprise one or more polymers selected from the group consisting of polyvinylpyrrolidone (PVP), acrylates and acrylic copolymers (e.g., Eudragit®), ethylcellulose (EC), hydroxypropylcellulose (HPC), polyvinyl alcohol, carboxymethylcellulose, and mixtures thereof. In some embodiments, the one or more polymers is selected from polyvinylpyrrolidone (PVP), hydroxypropylcellulose (HPC) and mixtures thereof. In some embodiments, the one or more polymers is polyvinyl alcohol or carboxymethylcellulose.

In some embodiments, the electrospun fibers of the present disclosure comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, polycaprolactone (PCL), dextran and gelatin. In some embodiments, the electrospun fibers comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the electrospun fibers of the present disclosure comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, the one or more polymers is selected from the group consisting of polyvinylpyrrolidone (PVP), acrylates and acrylic copolymers and mixtures thereof.

In some embodiments, the one or more polymers is polyvinylpyrrolidone (PVP), an ammonio methacrylate copolymer type B, or mixtures thereof.

In some embodiments, the one or more polymers comprise: (a) polyvinylpyrrolidone and an ammonio methacrylate copolymer; (b) polyvinylpyrrolidone, an ammonio methacrylate copolymer and polyethylene glycol; (c) polyvinylpyrrolidone, an ammonio methacrylate copolymer and polyethylene oxide; (d) polyvinylpyrrolidone, an ammonio methacrylate copolymer and dextran; (e) polyvinylpyrrolidone and gelatin; (f) polyvinylpyrrolidone, an ammonio methacrylate copolymer and gelatin; (g) polyvinylpyrrolidone and polyacrylate; (h) polyvinylpyrrolidone and sodium polyacrylate; (i) polyvinylpyrrolidone, an ammonio methacrylate copolymer and polyacrylate; (j) polyvinylpyrrolidone, an ammonio methacrylate copolymer and sodium polyacrylate; (k) polyvinylpyrrolidone and polyvinyl alcohol; (1) polyvinylpyrrolidone and polyethylene glycol; (m) polyvinylpyrrolidone, an ammonio methacrylate copolymer and polyvinyl alcohol; (n) polyvinylpyrrolidone and polyethylene oxide; or (o) polyvinylpyrrolidone and dextran.

In some embodiments, the one or more polymers comprise polyvinylpyrrolidone and ammonio methacrylate copolymer. In some embodiments, the weight ratio of polyvinylpyrrolidone to ammonio methacrylate copolymer in the electrospun fibers is about 0.1 to about 10, including, about 0.1, about 0.25, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to ammonio methacrylate copolymer is about 0.5 to about 5. In some embodiments, the weight ratio of polyvinylpyrrolidone to ammonio methacrylate copolymer is about 0.8 to about 2. In some embodiments, the weight ratio of polyvinylpyrrolidone to ammonio methacrylate copolymer is about 0.5 to about 1.5.

In some embodiments, the one or more polymers comprise polyvinylpyrrolidone and polyethylene glycol. In some embodiments, the weight ratio of polyvinylpyrrolidone to polyethylene glycol in the electrospun fibers is about 0.15 to about 10, e.g., including 0.15, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyethylene glycol is about 0.25 to about 5. In some embodiments, the weight ratio of polyvinylpyrrolidone to polyethylene glycol is about 0.8 to about 2. In some embodiments, the weight ratio of polyvinylpyrrolidone to polyethylene glycol is about 0.5 to about 1.5.

In some embodiments, the one or more polymers comprise polyvinylpyrrolidone and polyethylene oxide. In some embodiments, the weight ratio of polyvinylpyrrolidone to polyethylene oxide in the electrospun fibers is about 0.3 to about 10, including, about 0.3, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyethylene oxide is about 0.3 to about 5. In some embodiments, the weight ratio of polyvinylpyrrolidone to polyethylene oxide is about 0.8 to about 2. In some embodiments, the weight ratio of polyvinylpyrrolidone to polyethylene oxide is about 0.5 to about 1.5.

In some embodiments, the one more polymers comprise polyvinylpyrrolidone and dextran. In some embodiments, the weight ratio of polyvinylpyrrolidone to dextran in the electrospun fibers is about 0.3 to about 10, e.g., about 0.3, about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to dextran is about 0.3 to about 5. In some embodiments, the weight ratio of polyvinylpyrrolidone to dextran is about 0.8 to about 2. In some embodiments, the weight ratio of polyvinylpyrrolidone to dextran is about 0.5 to about 1.5.

In some embodiments, the one or more polymers comprise polyvinylpyrrolidone and gelatin. In some embodiments, the weight ratio of polyvinylpyrrolidone to gelatin in the electrospun fibers is about 0.6 to about 10, e.g., about 0.6, about 0.8, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to gelatin is about 0.6 to about 5. In some embodiments, the weight ratio of polyvinylpyrrolidone to gelatin is about 1 to about 3. In some embodiments, the weight ratio of polyvinylpyrrolidone to gelatin is about 0.8 to about 2. In some embodiments, the weight ratio of polyvinylpyrrolidone to gelatin is about 0.5 to about 1.5.

In some embodiments, the one or more polymers comprise polyvinylpyrrolidone and polyvinyl alcohol. In some embodiments, the weight ratio of polyvinylpyrrolidone to polyvinyl alcohol in the electrospun fibers is about 0.5 to about 10, e.g., about 0.5, about 0.75, about 1, about 1.5, about 2, about 2.5, about 3, about 3.5, about 4, about 4.5, about 5, about 5.5, about 6, about 6.5, about 7, about 7.5, about 8, about 8.5, about 9, about 9.5, or about 10, including all ranges and values therebetween. In some embodiments, the weight ratio of polyvinylpyrrolidone to polyvinyl alcohol is about 0.5 to about 5. In some embodiments, the weight ratio of polyvinylpyrrolidone to polyvinyl alcohol is about 1 to about 3. In some embodiments, the weight ratio of polyvinylpyrrolidone to polyvinyl alcohol is about 0.8 to about 2. In some embodiments, the weight ratio of polyvinylpyrrolidone to polyvinyl alcohol is about 0.5 to about 1.5.

In some embodiments, the one or more polymers comprise polyvinylpyrrolidone, an ammonio methacrylate copolymer and polyethylene glycol. In some embodiments, the weight ratio of polyvinylpyrrolidone to ammonio methacrylate copolymer to polyethylene glycol in the electrospun fibers is about 1:0.5:0. 1 to about 1:2: 6, including all ranges and values therebetween.

In some embodiments, the one or more polymers comprise polyvinylpyrrolidone, an ammonio methacrylate copolymer and polyethylene oxide. In some embodiments, the weight ratio of polyvinylpyrrolidone to ammonio methacrylate copolymer to polyethylene oxide in the electrospun fibers is about 2:1: 1 to about 2:2. 5:4, including all ranges and values therebetween.

In some embodiments, the one or more polymers comprise polyvinylpyrrolidone, an ammonio methacrylate copolymer and dextran. In some embodiments, the weight ratio of polyvinylpyrrolidone to ammonio methacrylate copolymer to dextran in the electrospun fibers is about 1:0.5:0.1 to about 1:2: 3, including all ranges and values therebetween.

In some embodiments, the one or more polymers comprise polyvinylpyrrolidone, an ammonio methacrylate copolymer and gelatin. In some embodiments, the weight ratio of polyvinylpyrrolidone to ammonio methacrylate copolymer to gelatin in the electrospun fibers is about 1:0.5:0.1 to about 1:2: 1.5, including all ranges and values therebetween.

In some embodiments, the one or more polymers comprise polyvinylpyrrolidone, an ammonio methacrylate copolymer and polyvinyl alcohol. In some embodiments, the weight ratio of polyvinylpyrrolidone to ammonio methacrylate copolymer to polyvinyl alcohol in the electrospun fibers is about 1:0.5:0.1 to about 1:2: 2, including all ranges and values therebetween.

In some embodiments, the electrospun fibers of the present disclosure comprise one or more polymers (as described herein), including hydrophilic polymers, which provide various desirable properties, e.g., providing for rheological properties suitable for electrospinning, physical properties (e.g., strength, flexibility, etc.) to provide a patch that is sufficiently durable for handling during manufacturing and use and sufficiently flexible such that it can conform to a mucosal surface, and a suitable degree of hydrophilicity for application to mucosal surfaces. Such polymers can provide bioadhesive properties to aid in adhesion to a mucosal surface. In some embodiments, one or more additional polymers can be added to improve bioadhesion. In some embodiments, the bioadhesive substance is selected from the group consisting of dextran, polyethylene oxide (PEO), alginate, tragacanth, carrageenan, pectin, gelatin, guar, xanthan, gellan, methylcellulose, hydroxypropylmethylcellulose (HPMC), carboxymethylcellulose and alkali salts thereof, polymers of acrylic acids (PAA derivatives), chitosan, lectins, thiolated polymers, polyox WSR, PAA-co-PEG (PEG is polyethylene glycol), and mixtures thereof. In some embodiments, the bioadhesive substance is PEO.

In some embodiments, the electrospun fibers of the present disclosure comprise polyvinylpyrrolidone and acrylic copolymers (e.g., Eudragit® copolymers), in combination with polyethylene oxide polymers. Such electrospun fiber compositions are described, for example, in U.S. Pat. No. 10,052,291, US Patent Publication Nos. 2019/0254985, and 2019/0254986, 2019/0351662.

When polyvinylpyrrolidone is used as a polymer in the electrospun fibers, it can be used in a grade having an approximate molecular weight of from 2,500 Da to 3,000,000 Da. PVP can be purchased as Kollidon® having a variety of molecular weight ranges as shown below.

| Kollidon ® | Weight average molecular weight M_{w} |
|---|---|
| 12PF | 2,000-3,000 |
| 17PF | 7,000-11,000 |
| 25 | 28,000-34,000 |
| 30 | 44,000-54,000 |
| 90F | 1,000,000-1,500,000 |

Ethylcellulose is sold under the trademark ETHOCEL™ (Dow Chemical Company) and is available in different grades. Dependent on its ethoxyl content, ethylcellulose may have different softening point and melting point temperatures. Ethylcellulose is also produced in a number of different viscosities (see Table below).

| | | Ethoxyl | Ethoxyl |
|---|---|---|---|
| Product | Viscosity | content % | content % |
| viscosity | range | Standard | Medium |
| designation | mPa*s | 48.0-49.5 | 45.0-46.5 |
| 4 | 3-5.5 | ETHOCEL Std. 4 | |
| 7 | 6-8 | ETHOCEL Std. 7 | |
| 10 | 9-11 | ETHOCEL Std. 10 | |
| 14 | 12.6-15.4 | ETHOCEL Std. 14 | |
| 20 | 18.22 | ETHOCEL Std. 20 | |
| 45 | 41.49 | ETHOCEL Std. 45 | |
| 50 | 45-55 | | ETHOCEL Med. 50 |
| 70 | 63-77 | | ETHOCEL Med. 70 |
| 100 | 90-110 | ETHOCEL Std. 100 | ETHOCEL Med. 100 |
| 200 | 180-220 | ETHOCEL Std. 200 | |
| 300 | 270-330 | ETHOCEL Std. 300 | |
| 350 | 250-385 | ETHOCEL Std. 4 | |

Acrylates and acrylic acid derivatives include polymethacrylates, methacrylate copolymers, acrylic copolymers and methacrylate polymers, and include acrylates sold as EUDRAGIT as well as acrylates/octaacrylamide sold as DERMACRYL 79. Non-limiting example of such acrylates are EUDRAGIT®E 12,5 (amino methacrylate copolymer), EUDRAGIT® E100 (amino methacrylate copolymer; basic butylated methacrylate copolymer), EUDRAGIT®E PO ((amino methacrylate copolymer), EUDRAGIT®L 100-55, EUDRAGIT®L 100 (methacrylic acid-methyl methacrylate copolymer 1:1), EUDRAGIT®S 100 (methacrylic acid-methyl methacrylate copolymer 1:2), EUDRAGIT®RL 100, EUDRAGIT®RL 100 (ammonio methacrylate copolymer type A), EUDRAGIT®RL PO, EUDRAGIT®RS100 (ammonio methacrylate copolymer type B), EUDRAGIT®RS PO. EUDRAGIT®E is a cationic polymer based on dimethylaminoethyl methacrylate and other neutral methacrylic acid esters: EUDRAGIT®L and S are methacrylic acid copolymers and are cationic copolymerization products of methacrylic acid and methyl methacrylate. EUDRAGIT®RL or RS is ammonio methacrylate copolymers synthesized from acrylic acid and methacrylic acid.

Carboxymethylcellulose is available in a broad selection of viscosity grades. In some embodiments, the viscosity of carboxymethylcellulose in the electrospun fibers ranges from 10 to 100,000 mPa*s. In some embodiments, the carboxymethylcellulose is a sodium salt.

Methylcellulose is sold under the name METHOCEL™ (Dow Chemical Company) and is available in a wide range of viscosity grades. In some embodiments, the viscosity grade of methylcellulose in the electrospun fibers is from less than about 3 to greater than about 100,000 mPA*s.

HPMC is sold under the names Methocel® and Klucel®. In some embodiments, HPMC of the electrospun fibers has an average molecular weight of from about 80,000 Da to about 140,000 Da.

In some embodiments, polyvinyl alcohol has a molecular weight ranging from about 20,000 Da to about 200,000 Da. In some embodiments, polyvinyl alcohol has a molecular weight ranging from about 100,000 Da to about 200,000 Da.

In some embodiments, polyethylene oxide (PEO) is used in grade having an approximate molecular weight of from about 100,000 Da to about 7,000,000 Da. In some embodiments, the average molecular weight of from about 700,000 Da to about 4,000,000 Da. Polyethylene oxide is sold under the name POLYOX™ (Dow Chemical Company) with molecular weights ranging from about 100,000 Da to about 7,000,000 Da.

In some embodiments, the PEO has a molecular weight of from about 100,000 Da to about 4,000,000 Da. In some embodiments, the PEO has a molecular weight of from about 100,000 Da to about 700,000 Da. In some embodiments, the PEO has a molecular weight of from about 100,000 Da to about 400,000 Da. In some embodiments, the PEO has a molecular weight of about 200,000 Da. In some embodiments, the PEO has a molecular weight greater than about 2,000,000 Da. In some embodiments, the PEO has a molecular weight of from about 2,000,000 Da to about 7,000,000 Da.

In some embodiments, dextran is used in grade having a molecular weight of from about 400,000 Da to about 2,000,000 Da. In some embodiments, dextran has a molecular weight of from about 500,000 Da to about 2,000,000 Da, e.g., about 700,000 Da to about 800,000 Da or from about 1,000,000 Da to about 2,000,000 Da.

In some embodiments, the one or more polymers are present in an amount ranging from about 30% to about 99.9% by weight of the electrospun fibers, e.g., about 30%, about 35%, about 40%, about 45%, about 50%, about 55%, about 60%, about 65%, about 70%, about 75%, about 80%, about 85%, about 90%, about 95%, or about 99.9%, including all ranges and values therebetween. In some embodiments, the one or more polymers are present in an amount ranging from about 50% to about 99.9% by weight of the electrospun fibers. In some embodiments, the one or more polymers are present in an amount ranging from about 75% to about 99.9% by weight of the electrospun fibers. In some embodiments, the one or more polymers are present in an amount ranging from about 75% to about 95% by weight of the electrospun fibers. In some embodiments, the one or more polymers are present in an amount ranging from about 75% to about 90% by weight of the electrospun fibers.

As disclosed herein, the electrospun fibers of the present disclosure comprise a therapeutically effective amount of an active agent. In some embodiments, the active agent is an anti-inflammatory agent and/or an immunosuppressant.

In some embodiments, the anti-inflammatory/immunosuppressant is a corticosteroid. In some embodiments, the corticosteroid is selected from the group consisting of amcinonide, betamethasone, budesonide, clobetasol, clobetasone, cortisone, desonide, desoxycortisone, desoximethasone, dexamethasone, diflucortolon, diflorasone, flucortisone, flumethasone, flunisolide, fluocinonide, fluocinolon, fluorometholone, fluprednisolone, flurandrenolide, fluticasone, halcinonide, halobetasol, hydrocortisone, meprednisone, methylprednisone, mometasone, paramethasone, prednicarbate, prednisone, prednisolone and triamcinolone or a pharmaceutically acceptable ester or acetonide thereof. In some embodiments, the steroid is selected from the group consisting of betamethasone, budesonide, clobetasol, clobetasone, desoximethasone, diflucortolon, diflorasone, fluocinonide, fluocinolon, halcinonide, halobetasol, hydrocortisone, mometasone and triamcinolone or a pharmaceutically acceptable ester thereof. In some embodiments, the corticosteroid is selected from the group consisting of diflorasone diacetate, betamethasone dipropionate, mometasone furoate and clobetasol propionate. In some embodiments, the corticosteroid is mometasone furoate.

In some embodiments, the anti-inflammatory agent is a non-steroidal anti-inflammatory active agent (NSAID). In some embodiments, the NSAID is selected from the group consisting of diclofenac, etodolac, fenoprofen, flurbiprofen, ibuprofen, indomethacin, meclofenamate, mefenamic acid, meloxicam, nabumetone, naproxen, oxaprozin, piroxicam, sulindac, and tolmetin

In some embodiments, the immunosuppressant is a calcineurin inhibitor. In some embodiments, the calcineurin inhibitor is selected from the group consisting of tacrolimus, picrolimus, pimecrolimus, cyclosporine, or voclosporin. In some embodiments, the calcineurin inhibitor is selected from the group consisting of tacrolimus, picrolimus, and cyclosporine.

In some embodiments, the immunosuppressant is an interleukin inhibitor, TNF-a inhibitor, or selective immunosuppressant. In some embodiments, the interleukin inhibitor is rilonacept, secukinumab, ixekizumab, brodalumab, ustekinumab, risankizumab, guselkumab, tildrakizumab. In some embodiments, the TNF-α inhibitor is etanercept or infliximab. In some embodiments, the immunosuppressant is pomalidomide, methotrexate, azathioprine, or lenalidomide.

In some embodiments, the electrospun fibers of the present disclosure comprise an analgesic selected from the group consisting of local anesthetics (e.g., lidocaine, benzocaine, and the like), acetaminophen, nonsteroidal anti-inflammatory active agents (NSAIDs), benzamidine hydrochloride, etomidate, ketamine, propofol, gabapentin, tramadol, and pregabalin. In some embodiments, the analgesic is selected from the group consisting of lidocaine, acetaminophen, nonsteroidal anti-inflammatory active agents (NSAIDs, e.g., aspirin, naproxen, ibuprofen, diclofenac, etc.), etomidate, ketamine, propofol, gabapentin, tramadol, and pregabalin. In some embodiments, the NSAID is selected from the group consisting of diclofenac, diflunisal, etodolac, ibuprofen, indomethacin, ketoprofen, ketorolac, nabumetone, naproxen, oxaprozin, piroxicam, salsalate, sulindac, and tolmetin. In some embodiments, the NSAID is selected from the group consisting of aspirin, naproxen, ibuprofen, and diclofenac. In some embodiments, the analgesic is a topical analgesic. In some embodiments, the analgesic is selected from the group consisting of lidocaine, benzocaine, prilocaine, xylocaine, tetracaine, capsaicin, menthol, and methyl salicylate. In some embodiments, the analgesic is lidocaine or articaine. In some embodiments, the analgesic is lidocaine.

In some embodiments, the active agent is about 0.01% to about 15.0% (wt./wt. %) of the electrospun fiber or fiber layer, e.g., about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, or about 15%, including all ranges and values therebetween. In some embodiments, the active agent is about 0.01% to about 10% (wt./wt. %) of the electrospun fiber or fiber layer. In some embodiments, the active agent is about 0.01% to about 5.0% (wt./wt. %) of the electrospun fiber or fiber layer. In some embodiments, the active agent is about 1% to about 10% (wt./wt. %) of the electrospun fiber or fiber layer. In some embodiments, the active agent is about 1% to about 5.0% (wt./wt. %) of the electrospun fiber or fiber layer.

In some embodiments, at least about 50% of an active agent disclosed herein is released from the electrospun fiber layer within about 120 min, about 90 min, about 60 min, or about 30 min after application of the patch to the target site. In some embodiments, at least about 50% of an active agent disclosed herein is released from the electrospun fiber layer within about 30 min after application. In some embodiments, complete or substantially complete (e.g., greater than 85%, greater than 90%, greater than 95%, or greater than 99%) active agent release from the electrospun fiber layer is achieved about 2-3 h after application

The present disclosure contemplates electrospun fibers combining any of the therapeutic active agent described herein with any of the aforementioned polymers.

In some embodiments, the electrospun fibers further comprise a plasticizer. In some embodiments, the plasticizers of the present disclosure include any pharmaceutically acceptable plasticizer known in the art having favorable organoleptic properties, e.g., no odor or poor taste.

In some embodiments, the plasticizer is a hydrophobic plasticizer. In some embodiments, the hydrophobic plasticizer is selected from the group consisting of acetyl triethyl citrate, tributyl citrate, triethylcitrate, castor oil, diacetylated monoglycerides, dibutyl sebacate, diethyl phthalate, triacetin, tributyrin, cellulose nitrate, or mixtures thereof. In some embodiments, the plasticizer is a hydrophilic plasticizer. In some embodiments, the hydrophilic plasticizer is selected from the group consisting of sorbitol, glycerol, polyethylene glycols (e.g., polyethylene glycol 1500), polyethylene glycol monomethyl ether, propylene glycol, or mixtures thereof.

In some embodiments, the plasticizer is a biodegradable polymer. In some embodiments, the biodegradable polymer is a polyester. In some embodiments, the biodegradable polymer is poly (lactic acid) or poly (lactide-co-glycolide).

In some embodiments, the plasticizer is a polymeric plasticizer. In some embodiments, the polymeric plasticizer has a molecular weight up to about 100,000 Da. In some embodiments, the polymeric plasticizer has a molecular weight from about 20,000 Da to about 100,000 Da, e.g., about 20,000 Da, about 25,000 Da, about 30,000 Da, about 35,000 Da, about 40,000 Da, about 45,000 Da, about 50,000 Da, about 55,000 Da, about 60,000 Da, about 65,000 Da, about 70,000 Da, about 75,000 Da, about 80,000 Da, about 85,000 Da, about 90,000 Da, about 95,000 Da, or about 100,000 Da, including all ranges and values therebetween. In some embodiments, the polymeric plasticizer has a molecular weight from about 100,000 Da to about 200,000 Da. In some embodiments, the polymeric plasticizer is a polyethylene glycol, polyethylene glycol monomethyl ether, polyesteramide, poly (lactic acid) or poly (lactide-co-glycolide). In some embodiments, the polymeric plasticizer is a polyethylene glycol, polyethylene glycol monomethyl ether, poly (lactic acid) or poly (lactide-co-glycolide). In some embodiments, the polymeric plasticizer is a polyethylene glycol or polyethylene glycol monomethyl ether.

In some embodiments, the plasticizer is an oligomeric plasticizer. In some embodiments, the oligomeric plasticizer has up to about 100 repeating units, up to about 50 repeating units, up to about 25 repeating units, or up to about 10 repeating units. In some embodiments, the oligomeric plasticizer has from about 10 to 100 repeating units.

In some embodiments, the plasticizer is a non-polymeric plasticizer. In some embodiments, the non-polymeric plasticizer is selected from the group consisting of citrate esters, fatty acid esters, sebacate esters, phthalate esters, and glycol derivatives. In some embodiments, the non-polymeric plasticizer is selected from the group consisting of triethyl citrate, acetyl triethyl citrate, tributyl citrate, butyl stearate, glycerol monostearate, stearyl alcohol, castor oil, mineral oil, diacetylated monoglycerides, dibutyl sebacate, diethyl phthalate, dibutyl phthalate, dioctyl phosphate, sorbitol, glycerol, triacetin, tributyrin, propylene glycol, or mixtures thereof.

In some embodiments, the plasticizer comprises a blend of two or more plasticizers. In some embodiments, the plasticizer comprises and blend of a polymeric and non-polymeric plasticizer. In some embodiments, the blend comprises a non-polymeric plasticizer disclosed herein and one or more polymers of the electrospun fibers disclosed herein. In some embodiments, the blend comprises one or more polymers selected from the group consisting of hydroxypropyl methylcellulose, hydroxypropyl methylcellulose, and methacrylic acid copolymer plasticized with a non-polymeric plasticizer disclosed herein.

In some embodiments, the plasticizer is selected from the group consisting of citrate esters, fatty acid esters, sebacate esters, phthalate esters, and glycol derivatives.

In some embodiments, the plasticizer is selected from the group consisting of triethyl citrate, acetyl triethyl citrate, tributyl citrate, butyl stearate, glycerol monostearate, stearyl alcohol, castor oil, mineral oil, diacetylated monoglycerides, dibutyl sebacate, diethyl phthalate, dibutyl phthalate, dioctyl phosphate, sorbitol, glycerol, triacetin, tributyrin, cellulose nitrate, polyethylene glycol 1500, polyethylene glycol monomethyl ether, propylene glycol, or mixtures thereof. In some embodiments, the hydrophobic plasticizer is selected from the group consisting of acetyl triethyl citrate, tributyl citrate, triethylcitrate, castor oil, diacetylated monoglycerides, dibutyl sebacate, diethyl phthalate, triacetin, tributyrin, cellulose nitrate, or mixtures thereof. In some embodiments, the hydrophilic plasticizer is selected from the group consisting of sorbitol, glycerol, polyethylene glycols, polyethylene glycol monomethyl ether, propylene glycol, or mixtures thereof. In some embodiments, the plasticizer is dibutyl sebacate.

In some embodiments, the electrospun fibers comprise about 1% to about 30% by weight of the plasticizer, e.g., about 1%, about 2.5%, about 5%, about 7.5%, about 10%, about 12.5%, about 15%, about 17.5%, about 20%, about 22.5%, about 25%, about 27.5%, or about 30%. In some embodiments, the electrospun fibers comprise about 2.5% to about 30% by weight of the plasticizer. In some embodiments, the electrospun fibers comprise about 2.5% to about 25% by weight of the plasticizer. In some embodiments, the electrospun fibers comprise about 5% to about 20% by weight of the plasticizer. In some embodiments, the electrospun fibers comprise about 5% to about 10% by weight of the plasticizer. In some embodiments, the electrospun fibers comprise about 10% by weight of the plasticizer.

In some embodiments, the electrospun fibers of the present disclosure further comprise an absorption enhancer, which improves the absorption or permeation of active agent through the skin or mucosa. In some embodiments, the presence of an absorption enhancer improves sublingual absorption or permeation of a therapeutic active agent. In some embodiments, the presence of an absorption enhancer improves oromucosal absorption or permeation of a therapeutic active agent.

In some embodiments, the absorption enhancer is selected from the group consisting of a fatty acid, a non-ionic surfactant, a polycation, a thiolated polymer, a cyclodextrin, and a cell-penetrating peptide.

In some embodiments, the absorption enhancer is a fatty acid. In some embodiments, the fatty acid is selected from the group consisting of caproic acid, caprylic acid, capric acid, sodium caprate, lauric acid, stearic acid, oleic acid, linoleic acid, and linolenic acid.

In some embodiments, the absorption enhancer is a non-ionic surfactant. In some embodiments, the non-ionic surfactant is selected from the group consisting of polysorbate (e.g., polysorbate 20, polysorbate 40, polysorbate 60, and polysorbate 80), polyethylene glycol alkyl ether (e.g., polyethylene glycol dodecyl ether, polyethylene glycol hexadecyl ether, polyethylene glycol octadecyl ether and polyethylene glycol oleyl ether), polyoxyethylene alkyl ethers, (polyoxyethylene lauryl ether, polyoxyethylene oleyl ether, and polyoxyethylene stearyl ether), nonylphenoxypolyoxyethylene (NPPOE), laurate sucrose ester (SE), and sodium glycocholate.

In some embodiments, the absorption enhancer is a polycation. In some embodiments, the polycation is selected from the group consisting of a chitosan and its quaternary ammonium derivatives, a poly-L-arginine, an aminated gelatin, and cetylpyridinium chloride.

In some embodiments, the absorption enhancer is a thiolated polymer. In some embodiments, the thiolated polymer is selected from the group consisting of carboxymethyl cellulose-cysteine, polycarbophil (PCP)-cysteine, chitosan-thiobutylamidine, chitosan-thioglycolic acid, chitosan-thioethylamidine, chitosan iminothiolane, chitosan-glutathione conjugates, polyacrylic acid-cysteine, polymethacrylic acid-cysteine, and hyaluronic acid-L-cysteine.

In some embodiments, the absorption enhancer is a cyclodextrin. In some embodiments, the cyclodextrin is selected from the group consisting of a methylated β-cyclodextrin, hydroxypropyl-β-cyclodextrin, and sulphobutylether-β-cyclodextrin.

In some embodiments, the absorption enhancer is a cell-penetrating peptide. In some embodiments, the cell-penetrating peptide is selected from the group consisting of penetratin, Tat (YGRKKKRRQRRR), R6, R8, R9, pVEC (LLIILRRRIRKQAHAHSK), RRL helix (RRLRRLLRRLRRLLRRLR), shuffle (RWFKIQMQIRRWKNKK), and penetramax (KWFKIQMQIRRWKNKR).

In some embodiments, the patches of the present disclosure deliver an effective amount of a therapeutic active agent transdermally. Like transbuccal active agent delivery, transdermal delivery has several advantages including the ability to achieve steady-state active agent levels, bypass the hepatic first-pass metabolism, increase patient compliance and reduce gastrointestinal (GI) adverse effects.

In some embodiments, the efficiency of transdermal delivery is improved by addition of an absorption enhancer (i.e., a penetration enhancer) described herein. In some embodiments, the absorption enhancer is a fatty acid (e.g., oleic acid), terpene, surfactant, propylene glycol, pyrrolidone derivative (e.g., N-methyl-2-pyrroldone), carbamic acid derivative, dioxolone derivative, and dioxane derivative. In some embodiments, the absorption enhancer is a cell-penetrating peptide (e.g., penetratin), a skin-penetrating peptide (hyaluronic acid conjugated to phospholipid), or antimicrobial peptide (e.g., magainin, GIGKFLHSAKKFGKAFVG EIMNS). In some embodiments, the absorption enhancer comprises a nanocarrier, including, but not limited to liposomes, niosomes, transfersomes, and ethosomes. Absorption enhancers relevant to the present disclosure include those described in B. Chaulagain et al. “Passive Delivery of Protein Drugs Through Transdermal Route” Artificial Cells, Nanomedicine, and Biotechnology 2018, 46, 5471-5487, incorporated herein by reference in its entirety.

In some embodiments, the efficiency of transdermal delivery is improved by the application of heat to the skin, as described in S. Szunerits et al. “Heat: A Highly Efficient Skin Enhancer for Transdermal Drug Delivery” Front. Bioeng. Biotechnol. 2018, 6 (15), 1-13, incorporated herein by reference in its entirety. For example, in some embodiments, heat-assisted microporation is used to create transport channels in the skin for improved permeation of therapeutic active agents. In some embodiments, thermal ablation with lasers, based on the selective removal of the stratum corneum (SC) by localized microsecond heat pulses, is used to increase permeability of the skin's outer barrier to therapeutic active agents.

In some embodiments, to prevent premature detachment from the treatment area, as well as to provide other therapeutic benefits, the patches of the present disclosure comprise an impermeable layer.

In some embodiments, the impermeable layer is provided as a coating disposed on an active agent-containing layer. In some embodiments, the impermeable layer is co-spun with the active agent-containing, and is therefore integrated into the electrospun fibers.

In some embodiments, the impermeable layer is a backing layer. In some embodiments, the impermeable layer is a hydrophobic impermeable layer.

In some embodiments, the impermeable layer protects the active agent-containing layer(s) from moisture or saliva. In some embodiments, the impermeable layer protects the active agent-layer from being washed away from the application site, which results in the desired local therapeutic effect being reduced or eliminated. In some embodiments, the impermeable layer functions as an occlusive layer, which drives the penetration of active agent into the skin or mucosa.

In some embodiments, the impermeable layer comprises a hydrophobic polymer. In some embodiments, the hydrophobic polymer is selected from the group consisting of polyethylene-co-vinyl acetate, ethylcellulose, poly (caprolactone) (PCL), carbothane or polysoftane. In some embodiments, the hydrophobic polymer is PCL.

In some embodiments, the impermeable layer comprises one or more biodegradable polyesters. In some embodiments, the biodegradable polyester is an aliphatic polyester. In some embodiments, the biodegradable polyester is an aromatic polyester. In some embodiments, the impermeable layer comprises one or more polymers selected from the group consisting of poly (caprolactone), poly (glycolic acid), poly (lactic acid), poly (lactide-co-glycolide), poly (butylene succinate), poly (butylene succinate-co-adipate), poly (p-dioxanone), and poly (trimethylene carbonate). In some embodiments, the impermeable layer comprises one or more polymers selected from the group consisting of poly (caprolactone), poly (glycolic acid), poly (lactic acid), and poly (lactide-co-glycolide). In some embodiments, the impermeable layer comprises poly (caprolactone).

In some embodiments, the impermeable layer comprises polyethylene-co-vinyl acetate, ethylcellulose, poly (caprolactone), carbothane or polysoftane. In some embodiments, the impermeable layer comprises acrylates/octylacrylamide copolymer (sold under the name DERMACRL® 79), amino methacrylate copolymer

(EUDRAGIT®), dimethylaminoethyl methacrylate, methacrylate, methyl methacrylate (e.g., EUDRAGIT®E 100), or other acrylates.

In some embodiments, the impermeable layer comprises a plasticizer. In some embodiments, the plasticizer is a hydrophobic plasticizer disclosed herein. In some embodiments, the plasticizer is a hydrophilic plasticizer disclosed herein. In some embodiments, the plasticizer is a polymeric plasticizer disclosed herein. In some embodiments, the plasticizer is an oligomeric plasticizer disclosed herein. In some embodiments, the plasticizer is a non-polymeric plasticizer disclosed herein. In some embodiments, the plasticizer comprises a blend of two or more plasticizers. For example, in some embodiments, the plasticizer comprises a blend of a polymeric and non-polymeric plasticizer. In some embodiments, the plasticizer is selected from the group consisting of citrate esters, fatty acid esters, sebacate esters, phthalate esters, and glycol derivatives. In some embodiments, the plasticizer is selected from the group consisting of triethyl citrate, acetyl triethyl citrate, tributyl citrate, butyl stearate, glycerol monostearate, stearyl alcohol, castor oil, mineral oil, diacetylated monoglycerides, dibutyl sebacate, diethyl phthalate, dibutyl phthalate, dioctyl phosphate, sorbitol, glycerol, triacetin, tributyrin, cellulose nitrate, polyethylene glycol 1500, polyethylene glycol monomethyl ether, propylene glycol, or mixtures thereof. In some embodiments, the hydrophobic plasticizer is selected from the group consisting of acetyl triethyl citrate, tributyl citrate, triethylcitrate, castor oil, diacetylated monoglycerides, dibutyl sebacate, diethyl phthalate, triacetin, tributyrin, cellulose nitrate, or mixtures thereof. In some embodiments, the hydrophilic plasticizer is selected from the group consisting of sorbitol, glycerol, polyethylene glycols, polyethylene glycol monomethyl ether, propylene glycol, or mixtures thereof. In some embodiments, the plasticizer is dibutyl sebacate.

In some embodiments, the impermeable layer comprises about 1% to about 50% by weight of the plasticizer, e.g., about 1%, about 2.5%, about 5%, about 7.5%, about 10%, about 12.5%, about 15%, about 17.5%, about 20%, about 22.5%, about 25%, about 27.5%, about 30%, about 32.5%, about 35%, about 37.5%, about 40%, about 42.5%, about 45%, about 47.5%, or about 50%. In some embodiments, the impermeable layer comprises about 1% to about 35% by weight of the plasticizer. In some embodiments, the impermeable layer comprises about 2.5% to about 25% by weight of the plasticizer. In some embodiments, the impermeable layer comprises about 5% to about 20% by weight of the plasticizer. In some embodiments, the impermeable layer comprises about 5% to about 10% by weight of the plasticizer. In some embodiments, the impermeable layer comprises about 10% by weight of the plasticizer.

In some embodiments, the impermeable layer comprises about 5-70% by weight of the patch, e.g., about 5%, about 10%, about 15%, about 20%, about 25%, about 30%, about 35%, about 40%, about 45%, about 50%, about 55%, about 60%, about 65%, or about 70%, including all values and ranges therebetween, by weight of the patch. In some embodiments, the impermeable layer comprises about 5-50% by weight of the patch. In some embodiments, the impermeable layer comprises about 10-30% by weight of the patch.

In some embodiments, the impermeable layer of the present disclosure has an area density less than about 100 grams per square meter (gsm), less than about 95 gsm, less than about 90 gsm, less than about 85 gsm, less than about 80 gsm, less than about 75 gsm, less than about 70 gsm, less than about 65 gsm, less than about 60 gsm, less than about 55 gsm, less than about 50 gsm, less than about 45 gsm, less than about 40 gsm, less than about 35 gsm, less than about 30 gsm, less than about 25 gsm, less than about 20 gsm, less than about 15 gsm, less than about 10 gsm, or less than about 5 gsm, including all ranges and values therebetween. In some embodiments, the impermeable layer has an area density less than about 100 gsm. In some embodiments, the impermeable layer has an area density less than about 75 gsm. In some embodiments, the impermeable layer has an area density less than about 50 gsm. In some embodiments, the impermeable layer has an area density less than about 25 gsm. In some embodiments, the impermeable layer has an area density less than about 15 gsm. In some embodiments, the impermeable layer has an area density of from about 10 gsm to about 100 gsm. In some embodiments, the impermeable layer has an area density of from about 15 gsm to about 75 gsm. In some embodiments, the impermeable layer has an area density of from about 15 gsm to about 50 gsm. In some embodiments, the impermeable layer has an area density of from about 15 gsm to about 30 gsm. In some embodiments, the impermeable layer has an area density of from about 15 gsm to about 20 gsm. In some embodiments, the impermeable layer has an area density of about 17 gsm. In some embodiments, the area density of the impermeable layer is about the same as the electrospun fiber layer. In some embodiments, reducing the area density of the impermeable layer results in an increase in the flexibility of the patch. In some embodiments, a patch with increased flexibility is more comfortable after application to the treatment area of a subject in need thereof. In some embodiments, a patch with increased flexibility adheres to the anogenital area for a longer time period compared to a less flexible patch.

In some embodiments, the present disclosure provides methods of treating a disease or condition in a patient in need thereof, the method comprising applying an active agent-containing patch disclosed herein to an affected area of a patient in need thereof.

In some embodiments, the present disclosure provides a method of treating an inflammatory condition, an autoimmune disease, or cancer. In some embodiments, the present disclosure provides a method of treating an inflammatory condition. In some embodiments, the inflammatory condition is an inflammatory skin condition disclosed herein. In some embodiments, the inflammatory skin condition is a skin condition that affects the male or female genitalia. In some embodiments, the inflammatory condition is an inflammatory mucosal condition disclosed herein.

In some embodiments, the disease or condition is a vulvar skin disease or condition. In some embodiments, the disease or condition is selected from the group consisting of vulvar lichen sclerosus, vulvar lichen planus, vulvar psoriasis, vulvar dermatosis and vulvar lichen simplex chronicus. In some embodiments, the condition is vulvar lichen sclerosus. In some embodiments, the disease or condition is a male genital skin disease or condition. In some embodiments, the disease or condition is male genital lichen sclerosus. In some embodiments, the condition is lichen sclerosus of the glans penis.

In some embodiments, the disease or condition is selected from the group consisting of recurrent aphthous stomatitis, oral lichen planus, pemphigoid, pemphigus, oral mucositis, Bechet's disease, and Lipschutz ulcers.

In some embodiments, the affected area of the patient is the anogenital area. In some embodiments, the affected area of the patient is the male or female genitalia. In some embodiments, the affected area of the patient is the vulva, glans penis, or perianal area. In some embodiments, the affected area of the patient is the vulva. In some embodiments, the affected area of the patient is the glans penis. In some embodiments, the affected area of the patient is the perianal area. In some embodiments, the affected area of the patient is the oral mucosa.

In some embodiments, the patch is applied to a vulva lichen sclerosus (VLS) lesion on the vulva. In some embodiments, the patch is applied to a VLS lesion on the perineum. In some embodiments, the patch is applied to a lichen sclerosus lesion on the male genitalia. In some embodiments, the patch is applied to a lichen sclerosus lesion on the glans penis.

In some embodiments, an active agent-containing patch of the present disclosure is administered (i.e., applied) to the anogenital area of a patient. In some embodiments, an active agent-containing patch of the present disclosure is administered (i.e., applied) to the vulva of a patient. In some embodiments, an active agent-containing patch of the present disclosure is administered (i.e., applied) to the glans penis of a patient. In some embodiments, an active agent-containing patch of the present disclosure is administered (i.e., applied) to the perianal area of a patient.

In some embodiments, an active agent-containing patch of the present disclosure is administered (i.e., applied) to the mucosa of a patient. In some embodiments, the mucosa is the oral mucosa of a patient (i.e., transbuccal or transmucosal administration).

In some embodiments, a patch of the present disclosure is administered (i.e., applied) to the anogenital area, the mucosa, a lesion (such as a VLS lesion), or other affected area of a patient. The patch of the present disclosure provides protection to the affected area thereby promoting the natural healing process, with or without an active agent.

The patches of the present disclosure may be administered by, for example, placing the patch on an anogenital surface in need of treatment with the active agent-containing electrospun fiber layer contacting the surface. The patch may be applied by hand or using an applicator.

The patches of the present disclosure may be administered by, for example, placing the patch on an oral mucosal in need of treatment with the active agent-containing electrospun fiber layer contacting the surface. The patch may be applied by hand or using an applicator.

In some embodiments, a patch of the present disclosure is administered once a day. In some embodiments, the patch is administered twice a day. In some embodiments, the patch is administered three times a day. In some embodiments, the patch is administered four times a day. In some embodiments, the patch is administered once a day for a period of about 1-8 weeks. In some embodiments, the patch is administered twice a day for a period of about 1-8 weeks. In some embodiments, the patch is administered three times a day for a period of about 1-8 weeks. In some embodiments, the patch is administered four times a day for a period of about 1-8 weeks. In some embodiments, the patch is administered once a day for a period of about 2-4 weeks. In some embodiments, the patch is administered twice a day for a period of about 2-4 weeks. In some embodiments, the patch is administered three times a day for a period of 2-4 weeks. In some embodiments, the patch is administered four times a day for a period of 2-4 weeks. In some embodiments, the patch is administered once a day for at least a month. In some embodiments, the patch is administered twice a day for at least a month. In some embodiments, the patch is administered three times a day for at least a month. In some embodiments, the patch is administered four times a day for at least a month. In some embodiments, the patch is administered once a day for at least a week. In some embodiments, the patch is administered twice a day for at least a week. In some embodiments, the patch is administered three times a day for at least a week. In some embodiments, the patch is administered four times a day for at least a week. In some embodiments, the patch is administered to the patient as needed.

In one aspect, the present disclosure provides a method of preparing a patch as described herein, the process comprising (a) combining a solvent with one or more polymers and an active agent of the present disclosure to provide an electrospinning mixture; (b) electrospinning the electrospinning mixture of step (a) to provide electrospun fibers; and (c) attaching an impermeable backing layer to the electrospun fibers of step (b).

In some embodiments, the conductivity of the electrospinning mixture of step (a) is from about 150 uS/cm to about 600 uS/cm, e.g., about 150 uS/cm, 175 uS/cm, 200 uS/cm, about 225 uS/cm, about 250 uS/cm, about 275 uS/cm, about 300 uS/cm, about 325 uS/cm, about 350 uS/cm, about 375 uS/cm, about 400 uS/cm, about 425 uS/cm, about 450 uS/cm, about 475 uS/cm, about 500 uS/cm, about 525 uS/cm, about 550 uS/cm, about 575 uS/cm, or about 600 uS/cm, including all ranges and values therebetween. In some embodiments, the conductivity of the electrospinning mixture of step (a) is from about 150 uS/cm to about 300 uS/cm.

In some embodiments, the solvent of Step (a) is an aqueous alcoholic solvent. In some embodiments, the aqueous alcohol solvent comprises ethanol and water. In some embodiments, the aqueous alcohol solvent comprises about 20% to about 97% (vol./vol. %) of ethanol. In some embodiments, the aqueous alcohol solvent comprises about 40% to about 97% (vol./vol. %) of ethanol. In some embodiments, the aqueous alcohol solvent comprises about 80% to about 97% (vol./vol. %) of ethanol. In some embodiments, the aqueous alcohol solvent comprises about 50% to about 80% (vol./vol. %) of ethanol. In some embodiments, the aqueous alcohol solvent comprises about 50% (vol./vol. %) of ethanol. In some embodiments, the aqueous alcohol solvent comprises about 80% (vol./vol. %) of ethanol. In some embodiments, the aqueous alcohol solvent comprises about 97% (vol./vol. %) of ethanol.

In some embodiments, the aqueous alcoholic solvent comprises a mixture of ethanol and 2% acetic acid in phosphate-buffered saline. In some embodiments, the aqueous solvent comprises about 40% to about 80% ethanol and 20% of 2% acetic acid in phosphate-buffered saline (PBS) as the remainder. In some embodiments, the aqueous solvent comprises about 80% ethanol and about 20% of 2% acetic acid in phosphate-buffered saline (PBS).

In some embodiments, the solvent of Step (a) comprises a mixture of an alcohol and dichloromethane or chloroform. In some embodiments, the alcohol is ethanol, n-propanol, or n-butanol. In some embodiments, the alcohol is n-butanol. In some embodiments, the solvent of Step (a) is a mixture of n-butanol and chloroform. In some embodiments, the alkyl chloride is chloroform. In some embodiments, the solvent of Step (a) comprises a mixture of two solvents selected from the group consisting of dichloromethane, ethanol, isopropanol, and butanone. In some embodiments, the solvent of Step (a) comprises n-butanol and chloroform.

In some embodiments, the electrospun fibers comprise one or more polymers selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

In some embodiments, Step (a) further comprises the addition of a plasticizer disclosed herein.

In some embodiments, the backing layer of Step (c) further comprises a plasticizer disclosed herein.

### EXAMPLES

### Example 1

Mometasone Loading and Release From Electrospun Fibers

Preparation of Electrospun Fibers

Electrospinning solutions having the compositions provided in Table 1 were prepared by dissolving the appropriate amounts of PVP, Eudragit RS100 and PEO in a 50% by weight solution of pure ethanol and demineralized water. Mometasone furoate was dissolved in DMSO and added to the polymer solution as need to achieve the desired concentration. In the cases of Samples 3 and 4, 1.25% by weight dibutyl sebacate was also added. Electrospun fibers were then prepared from each of the four solutions as described in International Publication Nos. WO2015/189212, WO2017/085264, WO2018/133910, and WO2018/133909; U.S. Pat. No. 10,052,291; and U.S. Publication Nos. 2019/0254985, 2019/0254986, and 2019/0351662, which are hereby incorporated by reference in their entireties for all purposes.

Upon analysis, it was determined that the electrospun fibers of Samples 3 and 4 comprise about 7.35% by weight of dibutyl sebacate plasticizer.

TABLE 1
Compositions of Electrospinning Solutions of Samples 1-4.

| | Sample 1 | | Sample 2 | | Sample 3 | | Sample 4 | |
|---|---|---|---|---|---|---|---|---|
| | Rivelin 0.1% | | Rivelin 0.01% | | Rivelin-DS 0.1% | | Rivelin-DS 0.01% | |
| | mometasone | | mometasone | | mometasone | | mometasone | |
| PVP | 4 | wt % | 4 | wt % | 4 | wt % | 4 | wt % |
| Eudragit RS100 | 5 | wt % | 5 | wt % | 5 | wt % | 5 | wt % |
| PEO | 8 | wt % | 8 | wt % | 8 | wt % | 8 | wt % |
| Mometasone furoate | 0.017 | wt % | 0.0017 | wt % | 0.017 | wt % | 0.0017 | wt % |

| | | | | | | |
|---|---|---|---|---|---|---|
| Dibutyl sebacate | — | — | 1.25 | wt % | 1.25 | wt % |

Encapsulation Efficiency

Release of Mometasone from Electrospun Material in Methanol

Procedure: Approximately 5 cm2 of electrospun material from Samples 1˜4 was cut into smaller pieces (˜ 0.5 cm2 not exact) and weighed. The pieces of electrospun material was subsequently transferred to a 4 mL scintillation vial and mixed with 2 mL methanol. After approximately 20 hours of incubation at ambient temperature with end-over-end rotation, the amount of released mometasone was determined by means of LC-MS analysis. Each Sample was tested in duplicate to provide two results for each Sample. For example, Sample 1 was tested in duplicate to provide results numbered 1.1 and 1.2; Sample 2 was tested in duplicate to provide results numbered 2.1 and 2.2, etc.

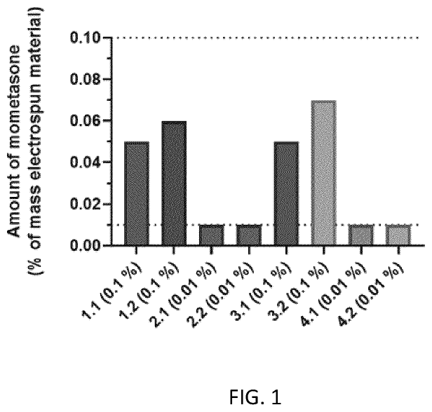

Results: FIG. 1 summarizes the total determined amount of mometasone recovered from the samples of electrospun material in methanol. For sample 1 (Rivelin 0.1%), an amount of 0.05% w/w and 0.06% w/w relative to mass of electrospun material was recovered. For sample 2 (Rivelin 0.01%), 0.01% w/w mometasone was recovered in both replicates. For sample 3 (Rivelin-DS 0.1%), 0.05 and 0.07% w/w mometasone was recovered in the two replicates. For sample 4 (Rivelin-DS 0.01%), an amount of 0.01% w/w of mometasone was recovered in both replicates.

Release of Mometasone from Electrospun Material in Buffer

Procedure: Approximately 10 cm2 of electrospun material was cut into smaller pieces (˜ 0.5 cm2 not exact) and weighed. The pieces of electrospun material were subsequently transferred to a 20 mL scintillation vial and mixed with 4 mL phosphate-buffered saline for 3 hours. At t=10, 20, 30, 60, 120 and 180 minutes, samples of 100 μL were taken and mixed with 100 μL methanol before analysis by means of LC-MS. The taken sample volume was immediately replaced with 100 μL blank phosphate-buffered saline. All samples were tested in duplicate.

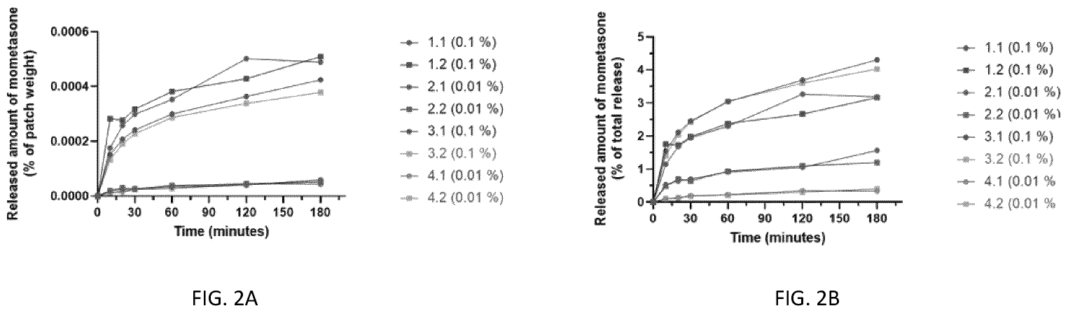

FIG. 2A and FIG. 2B summarize the release of mometasone from the samples of electrospun material over three hours in phosphate-buffered saline. The data provided as the amount of mometasone released as a percentage of patch weight (FIG. 2A) and as a percentage of total mometasone (FIG. 2B) show a greater release from Sample 1 and Sample 3 (FIG. 2A and FIG. 2B, top 4 lines). As indicated in FIG. 1, these samples contain a higher mometasone loading than Sample 2 and Sample 4. Depicting the release data relative to the average mometasone loading, showed a higher release from Sample 3 (FIG. 2B, top 2 lines; 1.25% DS), followed by sample 1 (FIG. 2B, lines 3 and 4 from top). The lowest release is observed for Sample 4 (FIG. 2B, bottom 2 lines).

### Study Conclusion

Between 0.05-0.07% mometasone was recovered from electrospun material that initially included 0.1 wt. % (Sample 1 and Sample 3), while 0.01% mometasone was recovered from electrospun material that initially included 0.01 wt. %. Controlling for the loading, the release of mometasone was higher from Sample 3 (Rivelin-DS 0.1%) followed by Sample 1 (Rivelin 0.1%). The lowest release was observed from Sample 4 (Rivelin-DS 0.01%).

### Example 2

Evaluation of Composition Flexibility

Objective: to determine if electrospun materials incorporating plasticizer (10% dibutyl sebacate) exhibit increased flexibility compared to a formulation without plasticizer.

The following electrospun compositions were prepared according to the methods referenced above and subjected to a flexibility test:

| | | Protective (Backing) |
|---|---|---|
| Composition | Formulation Bioadhesive Layer | Layer |
| A (monolayer) | Mass ratio PVP:RS100:PEO | — |
| | 1:1.25:2 | |
| B (monolayer; | Mass ratio PVP:RS100:PEO | — |
| modified) | 1:0.75:2 | |
| | 10% Dibutyl sebacate | |
| C (dual-layer)¹ | Mass ratio PVP:RS100:PEO | Polycaprolactone (PCL) + |
| | 1:1.25:2 | iron oxide powder |
| D (dual-layer; | Mass ratio PVP:RS100:PEO | Polycaprolactone (PCL) + |
| modified)¹ | 1:0.75:2 | iron oxide powder + 10% |
| | 10% Dibutyl sebacate | dibutyl sebacate |

¹Dual-layer compositions were calendared.

Test Procedure: Compositions A-D were analyzed using a Stable Micro Systems texture analyzer with a 3-point bend test rig. Six samples (7.5×2 cm) were cut from each electrospun composition and placed longitudinally on the rig with a separation between supports of 1.2 cm. The top probe applied a perpendicular force to the sample for a total time of 2 seconds at a speed of 1 mm/s. The force exerted when pushing and lifting was recorded

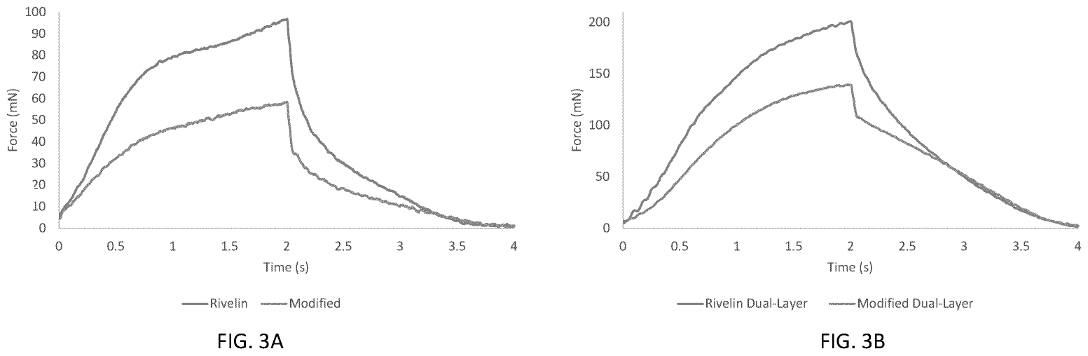

Results: FIG. 3A and FIG. 3B show the average of the applied force recorded for the six samples of each composition. For both Composition B and Composition D, the peak force (i.e., force applied at maximum deformation at 2 seconds) was lower compared to the respective unmodified composition without dibutyl sebacate. This suggests that by modifying monolayer and dual-layer electrospun materials with plasticizer, patches with increased flexibility can be produced.

### Example 3

Open Label Study

### INTRODUCTION

Vulvar lichen sclerosus (VLS) is a chronic inflammatory skin disease with a predilection for the anogenital area. Patients with VLS often have symptoms such as pruritus and pain.

It presents clinically as areas of atrophy, edema, fissures, erosion and/or thickened hyperkeratotic plaques. The initial treatment regimen consists of daily application of topical corticosteroids. Irritation from changes in microbiome and mechanical shear are thought to influence the disease severity.

An adhesive patch (PVP/RS100/PEO electrospun fibers and a PCL backing layer) has been developed to form a protective barrier over VLS lesions thereby potentially also contributing to pain relief.

Study Objectives

The present study was conducted in order to explore the adhesion and tolerability of plain patches (without medication) when applied directly to VLS lesions. Materials and Methods

The composition of the two-layer adhesive patch used in this study is as follows:
Electrospun fiber layer: 21% PVP, 26% RS100, and 42% PEO, each by weight of the patch; and
Impermeable layer: 10% PCL by weight of the patch.

Eligible subjects for the open label study were women (≥18 years) diagnosed with VLS which had signed an informed content. Pregnant or menstruating patients at the time of patch application were excluded.

The primary endpoint was applications of the patches and adhesion for ≥30 min. Secondary endpoints were frequency and intensity of adverse events collected during the investigation, subject's ability to apply patches correctly and understanding of the ‘instructions for use’ leaflet.

A visual analogue scale (VAS) for discomfort, pain, itching and burning was used at baseline, at 2 hours after application (or when the patch detached) and on day 2 (VAS 0-10, where 0=no symptom and 10=worst possible). At the same time physical examination grading redness, erosion and edema was performed (graded from 0 (no signs) to 4 (maximum)). Ten questions regarding the comfort, adhesion and the willingness to wear the patch containing medications was fulfilled day 2. Results

Twelve VLS patients aged 24 to 61 years (mean 55.1) were included. The majority had VLS for more than one year (<1 year (8.3%, n=1), 1-5 years (50%, n=6), 6-10 years (8.3%, n=1), >10 years (8.3%, n=1), NA (25%, n=3)). Baseline VLS symptoms by VAS scoring was as follows: pain (mean 2.6, range 0-7), itch (mean 3.8, range 0-10), burning (mean 3.3, range 0-8) and discomfort (mean 4.1, range 0-8.)

All patients were able to apply the plain patch. The mean adhesion time was 9.5 hon Day 1 and 10.0 h on Day 2. No AEs were reported. All subjects applied the patches correctly and were able to follow the leaflet instructions.

The measured VLS symptoms VAS scores (discomfort, pain, itching and burning) were lower 2 hours after application and on day 2 compared to baseline. In addition, for both redness, erosion and oedema, all subjects (except for 1) had scores of 0 or 1 on both day 1 and 2. For the physical examination of the mucosa and skin (vulva and anal area) 5 subjects (41.6%) had a shift from abnormal to normal for either skin or mucosa or both while no change were reported for the rest (58.3%) of the subjects. The overall response to the design of the patches was that they were easy and quick to apply.

### CONCLUSION

The primary objective to investigate the adhesion time of the plain patches to VLS lesions was met as all subjects reported a successful application with a mean adhesion time for both morning and evening of more than 9 hours. For all symptoms of VLS, the VAS scores were lower after patch application compared to prior to application indicating a reduction in the subjects' evaluation of the level of VLS symptoms after using the patch. No safety issues were reported and the patch were well tolerated when applied to sensitive VLS lesions.

What is claimed is:

1. A patch for treating a vulvar disease or condition, comprising:
(a) an electrospun fiber layer, wherein the electrospun fiber layer comprises electrospun fibers comprising:
one or more polymers; and
a therapeutically effective amount of an active agent, and
(b) an impermeable layer,
wherein after application to a patient in need thereof, the patch provides a therapeutically effective amount of the active agent for at least about 2 hours and wherein the patch stiffness is from about 250 mN/mm to about 450 mN/mm, as determined by a three-point bend test.

2. The patch of claim 1, wherein at least one of the electrospun fibers and the impermeable layer further comprises a plasticizer.

3. The patch of claim 1, wherein the electrospun fibers further comprise a plasticizer.

4. The patch of claim 1, wherein the impermeable layer comprises a plasticizer.

5. The patch of any one of claims 2-4, wherein the plasticizer is a hydrophobic plasticizer.

6. The patch of any one of claims 2-4, wherein the plasticizer is a hydrophilic plasticizer.

7. The patch of any one of claims 2-6, wherein the plasticizer is a polymeric plasticizer.

8. The patch of any one of claims 2-6, wherein the plasticizer is an oligomeric plasticizer.

9. The patch of any one of claims 2-8, wherein the plasticizer comprises a blend of two or more plasticizers.

10. The patch of any one of claims 2-4, wherein the plasticizer is selected from the group consisting of citrate esters, fatty acid esters, sebacate esters, phthalate esters, and glycol derivatives.

11. The patch of any one of claims 2-4, wherein the plasticizer is selected from the group consisting of triethyl citrate, acetyl triethyl citrate, tributyl citrate, butyl stearate, glycerol monostearate, stearyl alcohol, castor oil, mineral oil, diacetylated monoglycerides, dibutyl sebacate, diethyl phthalate, dibutyl phthalate, dioctyl phosphate, sorbitol, glycerol, triacetin, tributyrin, cellulose nitrate, polyethylene glycol 1500, polyethylene glycol monomethyl ether, propylene glycol, or mixtures thereof.

12. The patch of claim 5, wherein the hydrophobic plasticizer is selected from the group consisting of acetyl triethyl citrate, tributyl citrate, triethylcitrate, castor oil, diacetylated monoglycerides, dibutyl sebacate, diethyl phthalate, triacetin, tributyrin, cellulose nitrate, or mixtures thereof.

13. The patch of claim 6, wherein the hydrophilic plasticizer is selected from the group consisting of sorbitol, glycerol, polyethylene glycols, polyethylene glycol monomethyl ether, propylene glycol, or mixtures thereof.

14. The patch of any one of claims 2-4, wherein the plasticizer is dibutyl sebacate.

15. The patch of any one of claims 2-14, wherein the plasticizer is present in an amount ranging from about 1.0% to about 35% by weight of the patch.

16. The patch of claim 2 or 3, wherein the electrospun fibers comprise from about 1% to about 30% by weight of the plasticizer.

17. The patch of claim 3 or 4, wherein the impermeable layer comprises about 1% to about 50% by weight of the plasticizer.

18. The patch of any one of claims 1-17, wherein the one or more polymers comprise a hydrophilic polymer, a hydrophobic polymer, or mixture thereof.

19. The patch of any one of claims 1-18, wherein the one or more polymers is selected from the group consisting of polyvinylpyrrolidone, an ammonio methacrylate copolymer, polyethylene glycol, polyethylene oxide, polyacrylate, sodium polyacrylate, polyvinyl alcohol, dextran and gelatin.

20. The patch of any one of claims 1-19, wherein the one or more polymers comprise:
(a) polyvinylpyrrolidone and an ammonio methacrylate copolymer;
(b) polyvinylpyrrolidone, an ammonio methacrylate copolymer and polyethylene glycol;
(c) polyvinylpyrrolidone, an ammonio methacrylate copolymer and polyethylene oxide;
(d) polyvinylpyrrolidone, an ammonio methacrylate copolymer and dextran;
(e) polyvinylpyrrolidone and gelatin;
(f) polyvinylpyrrolidone, an ammonio methacrylate copolymer and gelatin;
(g) polyvinylpyrrolidone and polyacrylate;
(h) polyvinylpyrrolidone and sodium polyacrylate;
(i) polyvinylpyrrolidone, an ammonio methacrylate copolymer and polyacrylate;
(j) polyvinylpyrrolidone, an ammonio methacrylate copolymer and sodium polyacrylate;
(k) polyvinylpyrrolidone and polyvinyl alcohol;
(l) polyvinylpyrrolidone and polyethylene glycol;
(m) polyvinylpyrrolidone, an ammonio methacrylate copolymer and polyvinyl alcohol;
(n) polyvinylpyrrolidone and polyethylene oxide; or
(o) polyvinylpyrrolidone and dextran.

21. The patch of any one of claims 1-20, wherein the one or more polymers is selected from the group consisting of polyvinylpyrrolidone (PVP), acrylates and acrylic copolymers and mixtures thereof.

22. The patch of any one of claims 1-21, wherein the one or more polymers is polyvinylpyrrolidone (PVP), an ammonio methacrylate copolymer type B, or mixtures thereof.

23. The patch of claim 22, wherein the weight ratio of polyvinylpyrrolidone to ammonio methacrylate copolymer is about 0.8 to about 2.

24. The patch of any one of claims 1-18, wherein the one or more polymers is selected from the group consisting of the group consisting of polyethylene-co-vinyl acetate, ethylcellulose, poly (caprolactone) (PCL), carbothane or polysoftane.

25. The patch of any one of 1-24, wherein the impermeable layer comprises a hydrophobic polymer.

26. The patch of claim 25, wherein the hydrophobic polymer is selected from the group consisting of the group consisting of polyethylene-co-vinyl acetate, ethylcellulose, poly (caprolactone) (PCL), carbothane or polysoftane.

27. The patch of claim 25 or 26, wherein the hydrophobic polymer is PCL.

28. The patch of any one of claims 21-27, wherein the electrospun fiber layer further comprises a bioadhesive substance.

29. The patch of any one of claims 21-28, wherein the bioadhesive substance is selected from the group consisting of dextran, polyethylene oxide (PEO), alginate, tragacanth, carrageenan, pectin, gelatin, guar, xanthan, gellan, methylcellulose, hydroxypropylmethylcellulose (HPMC), carboxymethylcellulose and alkali salts thereof, polymers of acrylic acids (PAA derivatives), chitosan, lectins, thiolated polymers, polyox WSRA, PAA-co-PEG (PEG is polyethylene glycol), and mixtures thereof.

30. The patch of any one of claims 21-29, wherein the bioadhesive substance is PEO.

31. The patch of any one of claims 1-30, wherein the weight ratio of PVP: ammonio methacrylate copolymer B: PEO is from about 2:1: 1 to about 2:2.5:4.

32. The patch of any one of claims 1-31, wherein the average patch thickness prior to application is less than about 0.6 mm.

33. The patch of any one of claims 1-31, wherein the average patch thickness prior to application is less than about 0.3 mm.

34. The patch of any one of claims 1-31, wherein the area density of the electrospun fiber layer is less than about 125 grams per square meter (gsm).

35. The patch of any one of claims 1-31, wherein the area density of the electrospun fiber layer is less than about 75 gsm.

36. The patch of any one of claims 1-31, wherein the area density of the electrospun fiber layer is from about 15 gsm to about 75 gsm.

37. The patch of claims 1-36, wherein the area density of the electrospun fiber layer is greater than the impermeable layer.

38. The patch of any of claims 1-37, wherein the patch is semicircular.

39. The patch of any one of claims 1-38, wherein the vulvar disease or condition is selected from the group consisting of vulva lichen sclerosus, vulva lichen planus, vulva psoriasis, vulva dermatosis and vulva lichen simplex chronicus.

40. The patch of any one of claims 1-39, wherein the vulvar disease or condition is vulva lichen sclerosus.

41. The patch of any one of claims 1-40, wherein the active agent is an anti-inflammatory agent or an immunosuppressant.

42. The patch of any one of claims 1-41, wherein the anti-inflammatory agent/immunosuppressant is a corticosteroid.

43. The patch of claim 42, wherein the corticosteroid is selected from the group consisting of diflorasone diacetate, betamethasone dipropionate, mometasone furoate and clobetasol propionate.

44. The patch of claim 42, wherein the corticosteroid is mometasone furoate.

45. The patch of any one of claims 1-41, wherein the anti-inflammatory agent is a non-steroidal anti-inflammatory active agent (NSAID).

46. The patch of claim 45, wherein the NSAID is selected from the group consisting of diclofenac, etodolac, fenoprofen, flurbiprofen, ibuprofen, indomethacin, meclofenamate, mefenamic acid, meloxicam, nabumetone, naproxen, oxaprozin, piroxicam, sulindac, and tolmetin.

47. The patch of any one of claims 1-41, wherein the immunosuppressant is a calcineurin inhibitor.

48. The patch of claim 47, wherein the calcineurin inhibitor is cyclosporine, tacrolimus, or pimecrolimus.

49. The patch of any one of claims 1-41, wherein the immunosuppressant is an interleukin inhibitor, TNF-α inhibitor, or selective immunosuppressant.

50. The patch of any one of claims 1-41, wherein the immunosuppressant is pomalidomide, methotrexate, azathioprine, or lenalidomide.

51. The patch of any one of claims 1-50, wherein at least about 50% of the active agent is released from the electrospun fiber layer within about 30 min after application.

52. The patch of any one of claims 1-50, wherein complete active agent release from the electrospun fiber layer is achieved about 2-3 h after application.

53. The patch of claim 1, wherein the electrospun fibers comprise:
(a) a vulvar condition-treating active agent;
(b) one or more hydrophilic polymers;
(c) a bioadhesive substance; and
(d) an optional plasticizer
a hydrophobic layer, comprising:
(a) a hydrophobic polymer; and
(b) an optional plasticizer,
wherein the patch provides a therapeutically effective amount of the vulvar condition-treating active agent for at least about 2 hours after application to a vulvar lesion, and
wherein the patch stiffness is from about 250 mN/mm to about 450 mN/mm, as determined by a three-point bend test.

54. The patch of any one of claim 53, wherein the hydrophobic layer comprises a plasticizer.

55. The patch of any one of claims 53-54, wherein the hydrophobic layer comprises from about 5% to about 50% by weight of the plasticizer.

56. The patch of any one of claims 53-55, wherein the electrospun fibers comprise a plasticizer.

57. The patch of any one of claims 53-56, wherein the electrospun fibers comprise from about 5% to about 50% by weight of the plasticizer.

58. The patch of any one of claims 53-57, wherein the plasticizer is selected from the group consisting of acetyl triethyl citrate, tributyl citrate, triethylcitrate, castor oil, diacetylated monoglycerides, dibutyl sebacate, diethyl phthalate, sorbitol, glycerol, triacetin, tributyrin, cellulose nitrate, polyethylene glycol 1500, polyethylene glycol monomethyl ether, propylene glycol, or mixtures thereof.

59. The patch of any one of claims 53-58, wherein the plasticizer is dibutyl sebacate.

60. The patch of any one of claims 53-59, wherein the bioadhesive substance is selected from the group consisting of dextran, polyethylene oxide, alginate, tragacanth, carrageenan, pectin, gelatin, guar, xanthan, gellan, methylcellulose, hydroxypropylmethylcellulose (HPMC), carboxymethylcellulose and alkali salts thereof, polymers of acrylic acids (PAA derivatives), chitosan, lectins, thiolated polymers, polyox WSRA, PAA-co-PEG (PEG is polyethylene glycol), and mixtures thereof.

61. The patch of any one of claims 53-60, wherein the bioadhesive substance is polyethylene oxide.

62. The patch of any one of claims 53-61, wherein the hydrophilic polymer is selected from the group consisting of polyvinylpyrrolidone (PVP), acrylates and acrylic copolymers and mixtures thereof.

63. The patch of any one of claims 53-62, wherein the hydrophilic polymer is polyvinylpyrrolidone, an ammonio methacrylate copolymer type B, or mixtures thereof.

64. The patch of any one of claims 53-63, wherein the hydrophobic polymer is selected from the group consisting of polyethylene-co-vinyl acetate, ethylcellulose, poly (caprolactone) (PCL), carbothane or polysoftane.

65. The patch of any one of claims 53-64, wherein the hydrophobic polymer is PCL.

66. The patch of any one of claims 53-65, wherein the vulvar condition-treating active agent is a corticosteroid, an immunosuppressant, and/or an anti-inflammatory active agent.

67. The patch of claim 66, wherein the vulvar condition-treating active agent is a corticosteroid/immunosuppressant selected from the group consisting of diflorasone diacetate, betamethasone dipropionate, mometasone furoate and clobetasol propionate.

68. The patch of claim 66, wherein the vulvar condition-treating active agent is a corticosteroid/immunosuppressant selected from the group consisting of pimecrolimus and tacrolimus.

69. The patch of any one of claims 53-68, wherein:
the hydrophilic polymer comprises polyvinylpyrrolidone and an ammonio methacrylate copolymer type B;
the bioadhesive substance is PEO;
the hydrophobic polymer is PCL; and
the plasticizer is dibutyl sebacate.

70. A method of treating lichen sclerosus in a patient in need thereof, the method comprising applying a patch of any one of claims 1-69 to a lesion on a patient in need thereof.

71. The method of claim 70, wherein the lichen sclerosus is vulvar lichen sclerosus or penile lichen sclerosus.

72. The method of claim 70 or 71, wherein the patch is applied once a day, twice a day, three times a day or four times a day.

73. The method of any one of claims 70-72, wherein the patch is applied to a VLS lesion on the vulva.

74. The method of any one of claims 70-72, wherein the patch is applied to a VLS lesion on the perineum.

75. The method of any one of claims 70-72, wherein the patch is applied to a lesion on the glans penis.
